(19) United States
(12) Patent Application Publication
(10) Pub. No.: US 2008/0078003 A1
(43) Pub. Date: Mar. 27, 2008

(54) Marker Assisted Selection for Transformation Traits in Maize

(75) Inventors: Zhao Zuo-Yu, Johnston, IA (US); Oscar S. Smith, Redwood City, CA (US); Bailin Li, Hockessin, DE (US); Dinakar Bhattramakki, Johnston, IA (US); Guoping G. Shu, Johnston, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignees: PIONEER HI-BRED INTERNATIONAL, INC., Johnston, IA (US); E.I. DU PONT DE NEMOURS AND COMPANY, Wilmington, DE (US)

(21) Appl. No.: 11/855,402

(22) Filed: Sep. 14, 2007

Related U.S. Application Data
(60) Provisional application No. 60/825,618, filed on Sep. 14, 2006.

(51) Int. Cl. A01H 5/00

(52) U.S. Cl. 800/275

(57) ABSTRACT

Methods for producing corn with increased transformability are provided. Markers for increased transformability are provided as well as their use to obtain corn plants with increased transformability. Locations on chromosomes that effect transformation efficiency of monocots are identified.

### CROSS REFERENCE TO RELATED APPLICATIONS

This application claims the benefit of, and hereby incorporates by reference, provisional application 60/825,618 filed Sep. 14, 2006.

### FIELD OF THE INVENTION

The present invention relates to the field of molecular markers and transformation.

### BACKGROUND OF THE INVENTION

Culturability of crop plants has been shown to vary with the germplasm used. Some varieties or lines are easier to culture and regenerate than others. In many instances plants with the best agronomic traits tend to exhibit poor culturing and regeneration characteristics while plants that are more easily cultured and regenerated often exhibit poor agronomic traits. Work by Armstrong and others (D. D. Songstad, W. L. Petersen, C. L. Armstrong, American Journal of Botany, Vol. 79, pp. 761-764, 1992) showed that it was possible to interbreed a more culturable, agronomically poor maize line (A188) with an agronomically desirable, less culturable line (B73) to produce a novel line with increased culturability and regeneration (Hi-II). Marker analysis of the line was carried out and identified several chromosomal regions that appeared to confer increased culturability on the less culturable genetic background.

### SUMMARY OF THE INVENTION

In one aspect, the present invention provides methods of breeding maize plants for increased transformability as well as the markers used to track enhanced transformability. In one embodiment, the invention provides a process for producing an agronomically elite and transformable maize plant, comprising the steps of producing a population of plants by introgressing a chromosomal locus mapping to chromosome 1, 2, 3, 4, 5, 6, 7, 8, 9, or 10 from a more transformable maize genotype into a less transformable maize genotype. In certain embodiments of the invention, the process for producing an agronomically elite and transformable corn plant also comprises introgressing at least one chromosomal locus mapping to chromosome bins 1.01, 1.02, 1.03, 2.01, 2.02, 2.03, 2.04, 3.01, 3.02, 3.03, 3.04 3.05, 4.07, 4.08, 4.09, 5.03, 5.05, 5.07, 5.08 6.01, 6.02, 6.03, 6.04, 6.05, 6.06, 6.07, 6.08, 6.09, 7.04, 7.05, 8.01, 8.03, 8.04, 8.05, 8.06, 8.07, 10.01, 10.02, 10.03 or 10.04 from a transformable variety into an agronomically elite variety.

### DETAILED DESCRIPTION OF THE INVENTION

Breeding is a traditional and effective means of transferring the traits of one plant to another plant. Marker assisted breeding is a means of enhancing traditional breeding and allowing for selection of biochemical, yield or other less visible traits during the breeding process. While breeding work has been carried out to improve plant culture and regeneration, very little research has been carried out to identify and breed for chromosomal regions that are linked with enhanced transformation characteristics.

Maize lines often differ in transformability and/or culturability. The efficiency at which transgenic plants are produced from any given maize genotype is variable. Lines that can efficiently produce transgenic plants tend to be agronomically poor (for example Hi-II) while lines with superior or desired agronomic traits are less efficient at producing transgenic plant. If a desired gene is introduced into an agronomically poor line, it is then commonly introgressed into an elite or superior line for testing such parameters as efficacy of the introduced gene as well as to test the effect of the gene on such traits as yield, kernel quality and plant phenotype. Thus, to enable meaningful performance testing in earlier generations, it would be advantageous to be able to introduce the genetic components into maize inbreds which have increased transformability along with superior agronomic traits.

The present invention overcomes this deficiency in the art by providing a method of breeding for maize varieties with enhanced ability to produce transgenic plants.

Transformation of elite maize inbreds is an important technology for developing maize inbreds and hybrids with improved agronomic traits. Hi-II maize has been used for maize transformation for a number of years because of its high transformability and good culturability. Hi-II is a hybrid. Non-homozygous plants used in developing transgenic traits are problematic. It is easier to determine the effects of a transgene when a uniform, homozygous, background is used in transgene development. Another disadvantage of using Hi-II in transformation is that it does not have the quality genetics that are present in current elite maize inbreds. When developing a transgenic product the transgene is moved into an elite background through cross pollination. After the initial cross, backcrossing is used to remove as much of the Hi-II deleterious genome as possible. This is a labor intensive and time consuming process. It would therefore be beneficial to have a homozygous maize variety that has an elite genotype while also maintaining high transformability. Knowledge of the markers, chromosomal regions and genes that result in increased transformability would be beneficial in obtaining an elite maize inbred that has enhanced transformability.

A plant line, such as a maize inbred or hybrid, is said to exhibit “enhanced transformability” if the transformation efficiency of the line is greater than a parental line under substantially identical conditions of transformation. Transformation efficiency is a measure of the number of transgenic plants regenerated relative to the number of units of starting material (for example, immature embryos, pieces of callus and the like) exposed to an exogenous DNA, regardless of the type of starting material, the method of transformation, or the means of selection and regeneration. Under the breeding and transformation conditions described herein, a line is considered to exhibit enhanced transformability if a parent line goes through the breeding process and the result is a maize line with higher transformation efficiency than the original parental line.

For lines that have a measurable transformability, e.g., 0.001% to 0.01% or more, enhanced transformability can be measured by a fold increase. Transformation efficiency of the progeny germplasm after breeding may be enhanced from about two-fold to about three-fold beyond the transformation efficiency of the parental line. Alternatively, the transformation efficiency of the progeny germplasm after breeding may be enhanced about three-fold to about five-fold beyond the transformation efficiency of the parental line. It is contemplated that transformation efficiencies of progeny lines after breeding may be increased about five-fold to about ten-fold, from about five-fold to twenty-fold, and from about five-fold to about fifty-fold, and even from about five-fold to about one hundred-fold beyond the transformation efficiency of the parental line. A line is considered to demonstrate enhanced transformability when, after marker assisted breeding and transformation testing as described in the instant invention, the line exhibits at least a two-fold increase in transformation efficiency over the parental line.

The present invention overcomes limitations in the prior art of maize transformation by providing a method of breeding for enhance transformability. It is advantageous that maize lines exhibiting poor transformation capabilities can be bred according to the methods disclosed herein to result in lines which show enhanced transformability. It is particularly advantageous that the method may be applied to elite lines to impart enhanced transformability in agronomically desirable germplasm. The invention also identifies particular chromosomal locations important for the T-DNA delivery, culturability, regeneration and transformation. The invention identifies markers that can be used to track particular chromosomal locations so that breeding for highly transformable elite lines can be achieved in an efficient manner.

The method of the present invention was demonstrated using doubled haploid lines obtained from the Hi-II maize line. Because Hi-II is a hybrid, the population of doubled haploids formed from its progeny will be segregating for genes that can be associated with high transformability. One of skill in the art will recognize that any genotypes that are highly transformable may also be used. Progeny from various generations were tested for efficiency of T-DNA delivery, culturability, regenerability and overall transformability. Marker analysis indicated that regions associated with chromosomes 1, 2, 3, 4, 5, 6, 7, 8, 9, and 10 were associated with the enhanced transformability phenotype. One may introduce an enhanced transformability trait into any desired maize genetic background, for example, in the production of inbred lines suitable for production of hybrids, any other inbred lines, maize lines with desirable agronomic characteristics, or any maize line possessing an increased transformability trait. Using conventional plant breeding techniques, one may breed for enhanced transformability and maintain the trait in an inbred by self or sib-pollination.

An embodiment of the present invention is the use of any number or combination of molecular markers located in bins 1.01, 1.02, 1.03, 2.01, 2.02, 2.03, 2.04, 3.01, 3.02, 3.03, 3.04 3.05, 4.07, 4.08, 4.09, 5.03, 5.05, 5.07, 5.08 6.01, 6.02, 6.03, 6.04, 6.05, 6.06, 6.07, 6.08, 6.09, 7.04, 7.05, 8.01, 8.03, 8.04, 8.05, 8.06, 8.07, 10.01, 10.02, 10.03 or 10.04 to breed for increased transformability. Another embodiment is to breed for improved transformation efficiency with the use of any number or any combination of molecular markers located 20 centimorgans either side of the following markers: MARKER D, BNLG1014, UMC1254, UMC2013, UMC1792, MARKER J, UMC2133, UMC1708, UMC2087, UMC1774, UMC1797, UMC1265, PHI453121, MARKER E, UMC2041, MARKER G, UMC1365, MARKER F, UMC2035, UMC2294, UMC1339, UMC1433, UMC1287, UMC1607, BNLG1828, UMC1701, UMC1254, UMC1119, BNLG1720, BNLG1520, UMC1458, UMC1174, UMC1167, MARKER B, UMC1662, UMC1895, UMC1142, UMC2036, UMC1792, UMC1225, BNLG386, UMC1153, UMC1229; UMC1195, UMC1114, UMC2059, MARKER H, UMC1910, UMC1170, UMC2341, UMC2346, BNGL619, UMC2131, PHI041, Marker A, UMC1991, UMC2245, UMC1934, PHI427434, UMC2305, UMC1642, UMC1125, UMC1858, MARKER C, Marker L, PHI314704, PHI333597, Marker M, Marker N, PHI445613, Marker O, Marker Q, Marker R, BNLG1160, BNLG1174, BNLG1189, BNLG1647, PH1053, PMG1, UMC1025, UMC1043, UMC1075, UMC1086, UMC1400, UMC1412, UMC1424, UMC1495, UMC1587, UMC1667, UMC1808, UMC1814, UMC1830, UMC1853, UMC1907, UMC1908, UMC1949, UMC1985, UMC2258, UMC2260, UMC2264, UMC2265. The embodiments include at least one and any combination of the markers located 10, 5, 3, 2, or 1 centimorgans to either side of the markers listed above. The embodiments also include at least one of the listed markers or any combination thereof.

Other embodiments of the invention include the use of markers located in bin 2.02, 2.03, 2.04, 3.01, 3.02, 3.04, 3.05, 3.06, 4.07, 4.08, 4.09, 6.05, 6.06, 8.01 and 8.05 to breed for improved callus type. Improved callus type can be faster growth of callus as well as an increase in the percentage of embryos or other tissue types forming type-II callus. Other embodiments of the invention include breeding for improved callus using molecular markers located 20 centimorgans either side of the following markers: UMC2260, UMC2265, UMC1400, UMC1254, UMC1774, Marker M, UMC1985, BNLG1160, UMC1949, UMC1667, UMC1043, PHI314704, UMC1114, BNLG1174, PMG1, PHI445613, UMC1424, UMC1075, BNLG1647, UMC2258, Marker R, UMC1495, Marker N, UMC1908, UMC1797, UMC1265, PHI453121, MARKER E, UMC2041, MARKER G, UMC1365, MARKER F, UMC2035, UMC2294, UMC1339, UMC1433, UMC1287, UMC1607, and BNLG1828. The embodiments include using at least one and any combination of the markers located 10, 5, 3, 2, or 1 centimorgans to either side of the listed markers. The embodiments also include using at least one of the listed markers or any combination thereof.

Other embodiments of the invention include the use of markers located in bin 1.01, 2.01, 5.07, 5.08, 7.04, 7.05, 8.04, 8.05, 8.06, 8.07, 10.3, and 10.04 to breed for improved plant regeneration. Other embodiments of the invention include breeding for improved plant regeneration using molecular markers located 20 centimorgans either side of the following markers: BNLG1014, UMC1254, UMC2013, UMC1792, MARKER J, UMC2133, UMC1708, UMC2087, MARKER A, UMC1991, UMC1774, UMC2245-TA, UMC1265, UMC1934, PHI427434, UMC2305, UMC1642, UMC1433, UMC1125, UMC1858, MARKER C, UMC1170, BNGL619, and UMC2131. The embodiments include using at least one and any combination of the markers located 10, 5, 3, 2, or 1 centimorgans to either side of the listed markers. The embodiments also include using at least one of the listed markers or any combination thereof.

Embodiments of the invention include using a marker located in bin 1.01, 1.02, 2.01, 2.02, 2.03, 2.04, 3.01, 3.02, 3.04, 4.08, 4.09, 5.03, 5.07, 5.08 6.01, 6.05, 6.06, 6.07, 6.08, 6.09, 7.04, 7.05, 8.03, 8.04, 8.05, 8.06, 8.07, 10.01, 10.02, or 10.03 or along with markers disclosed in U.S. patent application Ser. No. 10/455,229 (Publication No. US 2004/0016030, published Jan. 22, 2004) to introgress genes that increase transformability from a more transformable maize line into a less transformable maize line. Embodiments include using any marker identified in Tables 2A, 3A, 5A, 6A, or 7A to map traits associated with increased transformability and using them with the markers disclosed in U.S. patent application Ser. No. 10/455,229 to breed for a maize line with increased transformability.

Embodiments of the invention include a method of obtaining a maize plant with increased efficiency for T-DNA delivery comprising: a) crossing a first maize plant and a second maize plant wherein said first plant has higher efficiency for T-DNA delivery than said second plant; b) taking DNA from cells obtained from said cross or from cells of later filial generations of said cross and hybridizing with one or more markers from a group consisting of a marker located in bin 5.02, 5.03, 5.04 and; c) selecting a plant wherein said DNA hybridizes with one or more of the markers to obtain a plant with higher efficiency for T-DNA delivery when compared to the efficiency for T-DNA delivery of the second plant. Any markers used for increasing efficiency of T-DNA delivery located between and including markers umc1587 and bnlg653 on chromosome 5 are also embodiments of the invention. Any markers used for increasing efficiency of T-DNA delivery located between and including markers umc1587 and bnlg653 on chromosome 5 and used in combination with markers located between and including umc1908 and umc2265 on chromosome 3 are also embodiments of the invention.

Embodiments of the invention include a method of selecting at least one maize plant by marker assisted selection of a quantitative trait locus associated with an increase in T-DNA delivery into a maize cell wherein said quantitative trait locus is localized to a chromosomal interval defined by and including markers umc1587 and bnlg653 on chromosome 5, said method comprising testing at least one marker on said chromosomal interval for said quantitative trait locus; and selecting said maize plant comprising said quantitative trait locus.

Embodiments of the invention include method of selecting at least one maize plant by marker assisted selection of a first quantitative trait locus and a second quantitative trait locus associated with an increase in T-DNA delivery into a maize cell wherein said first quantitative trait locus is localized to a chromosomal interval defined by and including markers umc1587 and bnlg653 on chromosome 5; and a said second quantitative trait locus is localized to a chromosomal interval defined by and including markers umc1908 and umc2265 on chromosome 3; said method comprising testing for said first quantitative trait locus and said second quantitative trait locus; and selecting said maize plant comprising said first and second quantitative loci.

Embodiments of the invention include a method of obtaining a maize plant with increased callus growth comprising: a) crossing a first maize plant and a second maize plant wherein said first plant has a higher callus growth rate than said second plant; b) taking DNA from cells obtained from said cross or from cells of later filial generations of said cross and hybridizing with one or more markers from a group consisting of a marker located in bin 4.07, 4.08 and; c) selecting a plant wherein said DNA hybridizes with one or more of the markers to obtain a plant with higher callus growth rate when compared to the callus growth rate of the second plant. Any markers used for increased callus growth rate located between and including markers bnlg1189 and bnlg1043 on chromosome 4 are also embodiments of the invention. Any markers used for increased callus growth rate located between and including markers bnlg1189 and bnlg1043 on chromosome 4 and used in combination with markers located between and including umc1908 and umc2265 on chromosome 3 are also embodiments of the invention.

Increases in transformability can be at least a 2× increase, a 20% increase, a 30% increase, or a 50% increase. Increases in tissue culture response can be at least a 2× increase, a 10% increase, 20% increase, a 30% or a 50% increase in Type II callus formation verses no callus growth or Type I callus growth. Increases in regeneration can be at least a 2× increase, a 10% increase, 20% increase, a 30% or a 50% increase in regeneration ability verses callus that will not regenerate into a plant. The increases can be due to introgression of one or more, or any combination of markers disclosed from the more transformable maize plant to the less transformable maize plant.

Marker assisted introgression involves the transfer of a chromosome region defined by one or more markers from one genome to a second genome. An initial step in that process is the localization of the trait by gene mapping which is the process of determining the position of a gene relative to other genes and genetic markers through linkage analysis. The basic principle for linkage mapping is that the closer together two genes are on the chromosome; the more likely they are to be inherited together. Briefly, a cross can be made between two parents differing in the traits under study. Genetic markers can then be used to follow the segregation of traits under study in the progeny from the cross (often a backcross (BC1), F₂, or recombinant inbred population). Genetic markers can also be associated with the increased transformability using a heterogeneous population of doubled haploids derived from a cross between two different parents.

Although a number of important agronomic characters are controlled by a single region on a chromosome (also known as a locus) or a single gene having a major effect on a phenotype, many economically important traits, such as yield and some forms of disease resistance, are quantitative in nature and involve a few to many genes or loci. The term quantitative trait loci, or QTL, is used to describe regions of a genome showing qualitative or additive effects upon a phenotype. As used herein, QTL refers to a chromosomal region defined by heritable genetic markers. The current invention relates to the introgression in maize of genetic material, e.g., at QTL, which is capable of causing a plant to be more easily transformed.

QTLs related to plant tissue culture and regeneration have been identified in wheat (Ben Amer et al., Plant Breeding, 114:84-85, 1995; Ben Amer et al., Theor. Appl. Genet., 94:1047-1052, 1997), rice (Taguchi-Shiobara et al. Theor. Appl. Genet., 95:828-833, 1997; Takeuchi et al., Crop Sci. 40:245-247, 2000; Kwon et al., Molecules and Cells, 11:64-67, 2001; Kwon et al., Molecules and Cells, 12:103-106), Arabidopsis (Schiantarelli et al., Theor. Appl. Genet., 102:335-342, 2001), barley (Mano et al., Breeding Science, 46:137-142, 1996; Bregitzer and Campbell, Crop Sci., 41:173-179, 2001) and corn (Armstrong et al., Theor. Appl. Genet., 84:755-762, 1992; Murigneux et al., Genome 37:970-976, 1994). In general, it is believed that many QTLs or chromosomal regions contribute to the process of T-DNA delivery, plant culturability, the ability to form somatic embryos, and the ability to regenerate into fertile plants. Furthermore, different QTLs are believed to be involved in the various steps of plant tissue culture and plant regeneration. It is of further desirable interest to identify QTLs that contribute to enhanced transformability of a plant and thereby to be able to manipulate plant performance of crops, such as but not limited to, corn, wheat, rice and barley.

Early work by Armstrong et al. investigated the use of breeding (Armstrong et al., Maize Gen. Coop. Newsletter, March 1, 65:92-93, 1991) and marker analysis (Armstrong et al., Theor. Appl. Genet., 84:755-762, 1992) to generate maize lines that were considered to be more culturable and regenerable than the parental maize lines. Armstrong et al. used parental line B73, a difficult line to culture but agronomically desirable, and A188, a highly culturable but agronomically poor line. Through a series of backcrosses and self-crosses, a more highly culturable line, named the “Hi-II” germplasm line, was developed. In comparison to the parental B73 line, the Hi-II line was found to be relatively easy to culture and regenerate healthy plants. RFLP analysis of markers which appeared to be associated with the increased culturability were located on chromosomes 1, 2, 3 and 9. The use of markers suggested that chromosomal regions of A188 remained in the B73 background, presumably allowing for the increased culturability and regenerability of the progeny Hi-II line. Of particular interest in this work was the marker c595 located on chromosome 9; it was suggested that a major gene or genes linked with marker c595 promote callus formation and plant regeneration.

It will be understood to those of skill in the art that other probes which more closely map the chromosomal regions as identified herein could be employed to identify crossover events. The chromosomal regions of the present invention facilitate introgression of increased transformability from readily transformable germplasm, such as Hi-II, into other germplasm, preferably elite inbreds. Larger linkage blocks likewise could be transferred within the scope of this invention as long as the chromosomal region enhances the transformability of a desirable inbred. Accordingly, it is emphasized that the present invention may be practiced using any molecular markers which genetically map in similar regions.

A plant genetic complement can be defined by a genetic marker profile that can be considered a “fingerprint” of a genome. For purposes of this invention, markers are preferably distributed evenly throughout the genome to increase the likelihood they will be near a quantitative trait locus or loci (QTL) of interest.

A sample first plant population may be genotyped for an inherited genetic marker to form a genotypic database. As used herein, an “inherited genetic marker” is an allele at a single locus. A locus is a position on a chromosome, and allele refers to conditions of genes; that is, different nucleotide sequences, at those loci. The marker allelic composition of each locus can be either homozygous or heterozygous.

Formation of a phenotypic database by quantitatively assessing one or more numerically representable phenotypic traits can be accomplished by making direct observations of such traits on progeny derived from artificial or natural self-pollination of a sample plant or by quantitatively assessing the combining ability of a sample plant.

By way of example, a plant line is crossed to, or by, one or more testers. Testers can be inbred lines, single, double, or multiple cross hybrids, or any other assemblage of plants produced or maintained by controlled or free mating, or any combination thereof. For some self-pollinating plants, direct evaluation without progeny testing is preferred.

The marker genotypes are determined in the testcross generation and the marker loci are mapped. To map a particular trait by the linkage approach, it is necessary to establish a positive correlation between the inheritance of a specific chromosomal region and the inheritance of the trait. This may be relatively straightforward for simply inherited traits. In the case of more complex inheritance, such as with as quantitative traits, linkage will be much more difficult to discern. In this case, statistical procedures must be used to establish the correlation between phenotype and genotype. This will further necessitate examination of many offspring from a particular cross, as individual loci may have small contributions to an overall phenotype.

Coinheritance, or genetic linkage, of a particular trait and a marker suggests that they are physically close together on the chromosome. Linkage is determined by analyzing the pattern of inheritance of a gene and a marker in a cross. In order for information to be gained from a genetic marker in a cross, the marker must by polymorphic; that is, it must exist in different forms so that the chromosome carrying the mutant gene can be distinguished from the chromosome with the normal gene by the form of the marker it also carries. The unit of recombination is the centimorgan (cM). Two markers are one centimorgan apart if they recombine in meiosis once in every 100 times. The centimorgan is a genetic measure, not a physical one, but a useful rule of thumb is that 1 cM is equivalent to approximately 10⁶ bp.

During meiosis, pairs of homologous chromosomes come together and exchange segments in a process called recombination. The farther a genetic marker, is from a gene, the more chance there is that there will be recombination between the gene and the marker. In a linkage analysis, the coinheritance of marker and gene or trait are followed in a particular cross. The probability that their observed inheritance pattern could occur by chance alone, i.e., that they are completely unlinked, is calculated. The calculation is then repeated assuming a particular degree of linkage, and the ratio of the two probabilities (no linkage versus a specified degree of linkage) is determined. This ratio expresses the odds for (and against) that degree of linkage, and because the logarithm of the ratio is used, it is known as the logarithm of the odds, e.g. a LOD score. A LOD score equal to or greater than 3, for example, is taken to confirm that gene and marker are linked. This represents 1000:1 odds that the two loci are linked. Calculations of linkage are greatly facilitated by use of statistical analysis employing programs.

The genetic linkage of marker molecules can be established by a gene mapping model such as, without limitation, the flanking marker model reported by Lander and Botstein (Genetics, 121:185-199, 1989), and the interval mapping, based on maximum likelihood methods described by Lander and Botstein (1989), and implemented in the software package MAPMAKER/QTL (Lincoln and Lander, 1990). Additional software includes Qgene, Version 2. 23 (1996), Department of Plant Breeding and Biometry, 266 Emerson Hall, Cornell University, Ithaca, N.Y.). Use of Qgene software is a particularly preferred approach.

A maximum likelihood estimate (MLE) for the presence of a marker is calculated, together with an MLE assuming no QTL effect, to avoid false positives. A log₁₀ of an odds ratio (LOD) is then calculated as: LOD=log₁₀ (MLE for the presence of a QTL/MLE given no linked QTL). The LOD score essentially indicates how much more likely the data are to have arisen assuming the presence of a QTL than in its absence. The LOD threshold value for avoiding a false positive with a given confidence, say 95%, depends on the number of markers and the length of the genome. Graphs indicating LOD thresholds are set forth in Lander and Botstein (1989), and further described by Arms and Moreno-González, Plant Breeding, Hayward, Bosemark, Romagosa (eds.) Chapman and Hall, London, pp. 314-331, 1993).

Additional models can be used. Many modifications and alternative approaches to interval mapping have been reported, including the use non-parametric methods (Kruglyak and Lander, Genetics, 121:1421-1428, 1995). Multiple regression methods or models can be also be used, in which the trait is regressed on a large number of markers (Jansen et al., Theor. Appl. Genet., 91:33-37, 1995; Weber and Wricke, Advances in Plant Breeding, Blackwell, 1994). Procedures combining interval mapping with regression analysis, whereby the phenotype is regressed onto a single putative QTL at a given marker interval, and at the same time onto a number of markers that serve as ‘cofactors,’ have been reported by Jansen and Stam, (Genetics, 136:1447-1455, 1994) and Zeng, (Genetics, 136:1457-1468, 1994). Generally, the use of cofactors reduces the bias and sampling error of the estimated QTL positions (Utz and Melchinger, Biometrics in Plant Breeding, Proceedings of the Ninth Meeting of the Eucarpia Section Biometrics in Plant Breeding, The Netherlands, 1994), thereby improving the precision and efficiency of QTL mapping (Zeng, 1994). These models can be extended to multi-environment experiments to analyze genotype-environment interactions (Jansen et al., 1995).

A number of different markers are available for use in genetic mapping. These include RLFP restriction fragment length polymorphisms (RFLPs), isozymes, simple sequence repeats (SSRs or microsatellites) and single nucleotide polymorphisms (SNPs) These markers are known to those of skill in the arts of plant breeding and molecular biology.

Several genetic linkage maps have been constructed which have located hundreds of RFLP markers on all 10 maize chromosomes. Molecular maps based upon RFLP markers have been reported for maize by several researchers examining a wide variety of traits (Burr et al., Genetics 118:519-526, 1988; Weber and Helentjaris, Genetics, 121:583-590, 1989; Stuber et al., Genetics, 132:823-839, 1992; Coe, Maize Genetics Cooperation Newsletter, 66:127-159, 1992; Gardiner et al., Genetics, 134:917-930, 1993; Sourdille et al., Euphytica, 91:21-30, 1996). One of skill in the art will recognize that genetic markers in maize are well know to those of skill in the art and are updated on a regular basis on the world wide web agron.missouri.edu. Another, type of genetic marker includes amplified simple sequence length polymorphisms (SSLPs) (Williams et al., Nucl. Acids Res., 18:6531-6535, 1990) more commonly known as simple sequence repeats (SSRs) or microsatellites (Taramino and Tingey, Genome, 39(2):277-287, 1996; Senior and Heun, Genome, 36(5):884-889, 1993). SSRs are regions of the genome which are characterized by numerous dinucleotide or trinucleotide repeats, e.g., AGAGAGAG. As with RFLP maps, genetic linkage maps have been constructed which have located hundreds of SSR markers on all 10 maize chromosomes.

Genetic linkage maps constructed using publicly available SNP markers are also available. For example, 21 loci along chromosome 1 have been mapped using SNPs (Tenaillon et al., Proc. Natl. Acad. Sci. U.S.A., 98(16):9161-9166, 2001) and over 300 polymorphic SNP markers have been identified from approximately 700 expressed sequence tags or genes from a comparison of M017 and B73 (Bhattramakki et al., Maize Genetics Coop. Newsletter 74:54, 2000).

One of skill in the art would recognize that many types of molecular markers are useful as tools to monitor genetic inheritance and are not limited to isozymes, RFLPs, SSRs and SNPs, and one of skill would also understand that a variety of detection methods may be employed to track the various molecular markers. One skilled in the art would also recognize that markers of different types may be used for mapping, especially as technology evolves and new types of markers and means for identification are identified.

Means of performing genetic marker profiles using SSR polymorphisms are well known in the art. SSRs are genetic markers based on polymorphisms in repeated nucleotide sequences, such as microsatellites. A marker system based on SSRs can be highly informative in linkage analysis relative to other marker systems in that multiple alleles may be present. Another advantage of this type of marker is that, through use of flanking primers, detection of SSRs can be achieved, for example, by the polymerase chain reaction (PCR), thereby eliminating the need for labor-intensive Southern hybridization. The PCR detection is done by use of two oligonucleotide primers flanking the polymorphic segment of repetitive DNA. Repeated cycles of heat denaturation of the DNA followed by annealing of the primers to their complementary sequences at low temperatures, and extension of the annealed primers with DNA polymerase, comprise the major part of the methodology.

Following amplification, markers can be scored by electrophoresis of the amplification products. Scoring of marker genotype is based on the size of the amplified fragment, which may be measured by the number of base pairs of the fragment. While variation in the primer used or in laboratory procedures can affect the reported fragment size, relative values should remain constant regardless of the specific primer or laboratory used. When comparing lines it is preferable if all SSR profiles are performed in the same lab. The SSR analyses reported herein were conducted in-house at Pioneer Hi-Bred. An SSR service is available to the public on a contractual basis by DNA Landmarks in Saint-Jean-sur-Richelieu, Quebec, Canada.

Primers used for the SSRs reported herein are publicly available and may be found in the Maize Genetic Database on the World Wide Web at maizegdb.org (sponsored by the USDA Agricultural Research Service), in Sharopova et al. (Plant Mol. Biol., 48(5-6):463-481), Lee et al. (Plant Mol. Biol., 48(5-6); 453-461), or may be constructed from sequences if reported herein. Primers may be constructed from publicly available sequence information. Some marker information may also be available from DNA Landmarks. Primers for markers that are not previously publicly reported are reported below.

| Marker | | |
|---|---|---|
| Identification | Left Primer | Right Primer |
| Marker A | SEQ ID 1: | SEQ ID 2: |
| | GCTCCACATCTGCTTTCCCTGT | TGCTCCCTTTGCGCTTTTAGAG |
| Marker B | SEQ ID 3: | SEQ ID 4: |
| | GTCGACCTCTCCATATCACAG | GCTGCTGCATGCATAAGAA |
| Marker C | SEQ ID 5: | SEQ ID 6: |
| | TCCTTCAAAGGTTCAAAGGACA | ATGTTATGAAACCGTGGCTGA |
| Marker D | SEQ ID 7: | SEQ ID 8: |
| | CATGACCACGACCATGAGC | GCAGGCGTCTCCACCTTT |
| Marker F | SEQ ID 9: | SEQ ID 10: |
| | GCGGTCTCTCTTCCTCTTCTTT | ACGAGGGGAAGGAGACGTT |
| Marker F | SEQ ID 11: | SEQ ID 12: |
| | TAAGCAGAGGCTCGTGGC | CGGCTCCTACTTCATGTACGTC |
| Marker G | SEQ ID 13: | SEQ ID 14: |
| | GGTGCTGAGAGAGAGGGAGA | CTCGCTGTTGCCTTCAAA |
| Marker H | SEQ ID 15: | SEQ ID 16: |
| | GGTGAACTGGGGAACGAC | CTGTTGTACAAGCTCCATCGG |
| Marker J | SEQ ID 17: | SEQ ID 18: |
| | CATTGCTTTGCTTCTCTTTCCC | TTTGATTGAGCTCGATTCGTC |
| Marker K | SEQ ID 19: | SEQ ID 20: |
| | TCGGCATCTTACGGGCTT | CGACGCACGCAGACTTTT |
| Marker L | SEQ ID 21: | SEQ ID 22: |
| | TGTCGTAGTCGCGGAGAAA | TAAACGCGCGAGTGGAGT |
| Marker M | SEQ ID 23: | SEQ ID 24: |
| | AAGTTCGGGACACCACCG | GCTGTTGCCCATGACGAT |
| Marker N | SEQ ID 25: | SEQ ID 26: |
| | CATGGTCTGCCAGATCGC | GCTGCTCAGGTTGTTGCC |
| Marker O | SEQ ID 27: | SEQ ID 28: |
| | AACGACCAGAGAGACACGG | CCGCCCGCATAGAGGATA |
| Marker Q | SEQ ID 29: | SEQ ID 30: |
| | CCGGCAGATGTTTCGATG | GAGGAAAGGATCGGACGC |
| Marker R | SEQ ID 31: | SEQ ID 32: |
| | GACAAGGGCGACAAGTGG | AACATACCAAAGCAGAGCAACC |

Map information is provided by bin number as reported in the Maize Genetic Database for the IBM 2 and/or IBM 2 Neighbors maps. The bin number digits to the left of decimal point represent the chromosome on which such marker is located, and the digits to the right of the decimal represent the location on such chromosome. Map positions are also available on the Maize GDB for a variety of different mapping populations.

For purposes of this invention, inherited marker genotypes maybe converted to numerical scores, e.g., if there are 2 forms of an RFLP, or other marker, designated A and B, at a particular locus using a particular enzyme, then diploid complements converted to a numerical score, for example, are AA=2, AB=1, and BB=0; or AA=1, AB=0 and BB=1. The absolute values of the scores are not important. What is important is the additive nature of the numeric designations. The above scores relate to codominant markers. A similar scoring system can be given that is consistent with dominant markers.

Particular markers used for these purposes are not limited to the set of markers disclosed herein, but may include any type of marker and marker profile which provides a means of breeding for a corn line that has increased transformation efficiency, increased transgene insertion into the native DNA, increased tissue culture response, or increased regeneration efficiency.

The present invention provides a method to increase transformability by use of marker assisted breeding wherein a population of plants are selected for an enhanced transformability trait. The selection comprises probing genomic DNA for the presence of marker molecules that are genetically linked to an allele of a QTL associated with enhanced transformability in the maize plant, where the alleles of a quantitative trait locus are also located on linkage groups on chromosomes 1, 2, 3, 4, 5, 6, 7, 8, 9, and 10 of a corn plant. The molecular marker is a DNA molecule that functions as a probe or primer to a target DNA molecule of a plant genome.

An F₂ population is the first generation of selfing after the hybrid seed is produced. Recombinant inbred lines (RIL) (genetically related lines; usually >F₅, developed from continuously selfing F₂ lines towards homozygosity) can be used as a mapping population. Information obtained from dominant markers can be maximized by using RIL because all loci are homozygous or nearly so.

Backcross populations (e.g., generated from a cross between a desirable variety (recurrent parent) and another variety (donor parent) carrying a trait not present in the former) can also be utilized as a mapping population. A series of backcrosses to the recurrent parent can be made to recover most of its desirable traits. Thus a population is created consisting of individuals similar to the recurrent parent but each individual carries varying amounts of genomic regions from the donor parent. Backcross populations can be useful for mapping dominant markers if all loci in the recurrent parent are homozygous and the donor and recurrent parent have contrasting polymorphic marker alleles (Reiter et al., 1992).

Another useful population for mapping are a near-isogenic lines (NIL). NILs are created by many backcrosses to produce an array of individuals that are nearly identical in genetic composition except for the desired trait or genomic region can be used as a mapping population. In mapping with NILs, only a portion of the polymorphic loci are expected to map to a selected region. Mapping may also be carried out on transformed plant lines.

Many methods may be used for detecting the presence or absence of the enhanced transformability QTLs of the current invention. Particularly, genetic markers which are genetically linked to the QTLs defined herein will find use with the current invention. Such markers may find particular benefit in the breeding of maize plants with increased transformability. This will generally comprise using genetic markers tightly linked to the QTLs defined herein to determine the genotype of the plant of interest at the relevant loci. Examples of particularly advantageous genetic markers for use with the current invention will be RFLPs and PCR based markers such as those based on micro satellite regions (SSRs) or single nucleotide polymorphisms (SNPs). A number of standard molecular biology techniques are useful in the practice of the invention. The tools are useful not only for the evaluation of markers, but for the general molecular and biochemical analyses of a plant for a given trait of interest. Such molecular methods include, but are not limited to, template dependent amplification methods such as PCR or reverse transcriptase PCR, protein analysis for monitoring expression of exogenous DNAs in a transgenic plant, including Western blotting and various protein gel detection methods, methods to examine DNA characteristics including Southern blotting, means for monitoring gene expression such as Northern blotting, and other methods such as gel chromatography, high performance liquid chromatography and the like.

Breeding techniques take advantage of a plant's method of pollination. There are two general methods of pollination: self-pollination which occurs if pollen from one flower is transferred to the same or another flower of the same plant, and cross-pollination which occurs if pollen comes to it from a flower on a different plant. Plants that have been self-pollinated and selected for type over many generations become homozygous at almost all gene loci and produce a uniform population of true breeding progeny, homozygous plants. In development of suitable inbreds, pedigree breeding may be used. The pedigree breeding method for specific traits involves crossing two genotypes. Each genotype can have one or more desirable characteristics lacking in the other; or, each genotype can complement the other. If the two original parental genotypes do not provide all of the desired characteristics, other genotypes can be included in the breeding population. Superior plants that are the products of these crosses are selfed and are again advanced in each successive generation. Each succeeding generation becomes more homogeneous as a result of self-pollination and selection. Typically, this method of breeding involves five or more generations of selfing and selection: S₁→S2; S₂→S3; S₃→S4; S4→S5, etc. A selfed generation (S) may be considered to be a type of filial generation (F) and may be named F as such. After at least five generations, the inbred plant is considered genetically pure. Molecular markers disclosed can be used in at least one filial or a combination of filial generations, S₁, S₂, S₃, S₄, S₅, etc., in order to introgress genes from the more transformable line to the elite less transformable line.

Breeding may also encompass the use of double haploid, or dihaploid, crop lines.

Backcrossing transfers specific desirable traits, such as the increased transformability QTL loci of the current invention, from one inbred or non-inbred source to an inbred that lacks that trait. This can be accomplished, for example, by first crossing a superior inbred (A) (recurrent parent) to a donor inbred (non-recurrent parent), which carries the appropriate gene(s) for the trait in question (Fehr, 1987). The progeny of this cross are then mated back to the superior recurrent parent (A) followed by selection in the resultant progeny for the desired trait to be transferred from the non-recurrent parent. Such selection can be based on genetic assays, as mentioned below, or alternatively, can be based on the phenotype of the progeny plant. After five or more backcross generations with selection for the desired trait, the progeny are heterozygous for loci controlling the characteristic being transferred, but are like the superior parent for most or almost all other genes. The last generation of the backcross is selfed, or sibbed, to give pure breeding progeny for the gene(s) being transferred, in the case of the instant invention, loci providing the plant with enhanced transformability.

In one embodiment of the invention, the process of backcross conversion may be defined as a process including the steps of:

(a) crossing a plant of a first genotype containing one or more desired gene, DNA sequence, region, or element, such as the QTLs, markers, or chromosomal regions identified in the present invention, to a plant of a second genotype lacking said desired gene, DNA sequence or element;

(b) selecting one or more progeny plant containing the desired gene, DNA sequence, region, or element;

(c) crossing the progeny plant to a plant of the second genotype; and

(d) repeating steps (b) and (c) for the purpose of transferring said desired gene, DNA sequence, region, or element from a plant of a first genotype to a plant of a second genotype.

These steps can be with any combination or any number of genes, DNA sequences, regions, or elements, such as the QTLs, markers, or chromosomal regions identified in the present invention.

Introgression of a particular DNA element or set of elements into a plant genotype is defined as the result of the process of backcross conversion. A plant genotype into which a DNA sequence has been introgressed may be referred to as a backcross converted genotype, line, inbred, or hybrid. Similarly a plant genotype lacking said desired DNA sequence may be referred to as an unconverted genotype, line, inbred, or hybrid. During breeding, the genetic markers linked to enhanced transformability may be used to assist in breeding for the purpose of producing maize plants with increased transformability. It is to be understood that the current invention includes conversions comprising one, or any number of the QTLs, chromosomal regions or markers, of the present invention. Therefore, when the term enhanced transformability or increased transformability converted plant is used in the context of the present invention; this includes any conversions of that plant utilizing the identified markers or chromosomal regions identified in the present invention. Backcrossing methods can therefore be used with the present invention to introduce the enhanced transformability trait of the current invention into any inbred by conversion of that inbred with one, two, three, or any combination or any number of the enhanced transformability loci. The selection of a suitable recurrent parent is an important step for a successful backcrossing procedure. The goal of a backcross protocol is to alter or substitute a trait or characteristic in the original inbred. To accomplish this, one or more loci of the recurrent inbred is modified or substituted with the desired gene from the nonrecurrent parent, while retaining essentially all of the rest of the desired genetic, and therefore the desired physiological and morphological, constitution of the original inbred. The choice of the particular nonrecurrent parent will depend on the purpose of the backcross, which in the case of the present invention will be to add the increased transformability trait to improve agronomically important varieties. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred. In the case of the present invention, one may test the transformability of progeny lines generated during the backcrossing program as well as using marker assisted breeding to select lines based upon markers rather than visual traits.

Backcrossing may additionally be used to convert one or more single gene traits into an inbred or hybrid line having the enhanced transformability of the current invention. Many single gene traits have been identified that are not regularly selected for in the development of a new inbred but that can be improved by backcrossing techniques. Single gene traits may or may not be transgenic, examples of these traits include but are not limited to, male sterility, waxy starch, herbicide resistance, resistance for bacterial, fungal, or viral disease, insect resistance, male fertility, enhanced nutritional quality, industrial usage, yield stability and yield enhancement. These genes are generally inherited through the nucleus. Some known exceptions to this are the genes for male sterility, some of which are inherited cytoplasmically, but still act as single gene traits.

Direct selection may be applied where the single gene acts as a dominant trait. An example might be the herbicide resistance trait. For this selection process, the progeny of the initial cross are sprayed with the herbicide prior to the backcrossing. The spraying eliminates any plants which do not have the desired herbicide resistance characteristic, and only those plants which have the herbicide resistance gene are used in the subsequent backcross. This process is then repeated for all additional backcross generations.

The waxy characteristic is an example of a recessive trait. In this example, the progeny resulting from the first backcross generation (BC1) must be grown and selfed. A test is then run on the selfed seed from the BC1 plant to determine which BC1 plants carried the recessive gene for the waxy trait. In other recessive traits, additional progeny testing, for example growing additional generations such as the BC1S1 may be required to determine which plants carry the recessive gene.

The development of uniform corn plant hybrids requires the development of homozygous inbred plants, the crossing of these inbred plants, and the evaluation of the crosses. Pedigree breeding and recurrent selection are examples of breeding methods used to develop inbred plants from breeding populations. Those breeding methods combine the genetic backgrounds from two or more inbred plants or various other broad-based sources into breeding pools from which new inbred plants are developed by selfing and selection of desired phenotypes. The new inbreds are crossed with other inbred plants and the hybrids from these crosses are evaluated to determine which of those have commercial potential. A single cross hybrid corn variety is the cross of two inbred plants, each of which has a genotype which complements the genotype of the other. The hybrid progeny of the first generation is designated F₁. Preferred F₁ hybrids are more vigorous than their inbred parents. This hybrid vigor, or heterosis, is manifested in many polygenic traits, including markedly improved higher yields, better stalks, better roots, better uniformity and better insect and disease resistance. In the development of hybrids only the F₁ hybrid plants are sought. An F₁ single cross hybrid is produced when two inbred plants are crossed. A double cross hybrid is produced from four inbred plants crossed in pairs (A×B and C×D) and then the two F₁ hybrids are crossed again (A×B)×(C×D).

As a final step, maize breeding generally combines two inbreds to produce a hybrid having a desired mix of traits. Getting the correct mix of traits from two inbreds in a hybrid can be difficult, especially when traits are not directly associated with phenotypic characteristics. In a conventional breeding program, pedigree breeding and recurrent selection breeding methods are employed to develop new inbred lines with desired traits. Maize breeding programs attempt to develop these inbred lines by self-pollinating plants and selecting the desirable plants from the populations. Inbreds tend to have poorer vigor and lower yield than hybrids; however, the progeny of an inbred cross usually evidences vigor. The progeny of a cross between two inbreds is often identified as an F₁ hybrid. In traditional breeding F₁ hybrids are evaluated to determine whether they show agronomically important and desirable traits. Identification of desirable agronomic traits has typically been done by breeders' expertise. A plant breeder identifies a desired trait for the area in which his plants are to be grown and selects inbreds which appear to pass the desirable trait or traits on to the hybrid.

Hybrid plants having the increased transformability of the current invention may be made by crossing a plant having increased transformability to a second plant lacking the enhanced transformability. “Crossing” a plant to provide a hybrid plant line having an increased transformability relative to a starting plant line, as disclosed herein, is defined as the techniques that result in the introduction of increased transformability into a hybrid line by crossing a starting inbred with a second inbred plant line that comprises the increased transformability trait. To achieve this one would, generally, perform the following steps:

(a) plant seeds of the first inbred and a second inbred donor plant line that comprises the enhanced transformability trait as defined herein;

(b) grow the seeds of the first and second parent plants into plants that produce flowers;

(c) allow cross pollination to occur between the plants; and (d) harvest seeds produced on the parent plant bearing the female flower.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques, 4:320-334), electroporation (Riggs et al. (1986) Proc. Natl. Acad. Sci. USA, 83:5602-5606, Agrobacterium-mediated transformation (Townsend et al., U.S. Pat. No. 5,563,055), direct gene transfer (Paszkowski et al. (1984) EMBO J., 3:2717-2722), and ballistic particle acceleration (see, for example, Sanford et al., U.S. Pat. No. 4,945,050; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology, 6:923-926). Also see Weissinger et al. (1988) Ann. Rev. Genet., 22:421-477; Sanford et al. (1987) Particulate Science and Technology, 5:27-37 (onion); Christou et al. (1988) Plant Physiol., 87:671-674 (soybean); McCabe et al. (1988) Bio/Technology, 6:923-926 (soybean); Finer and McMullen (1991) In Vitro Cell Dev. Biol., 27P:175-182 (soybean); Singh et al. (1998) Theor. Appl. Genet., 96:319-324 (soybean); Datta et al. (1990) Biotechnology, 8:736-740 (rice); Klein et al. (1988) Proc. Natl. Acad. Sci. USA, 85:4305-4309 (maize); Klein et al. (1988) Biotechnology, 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol., 91:440-444 (maize); Fromm et al. (1990) Biotechnology, 8:833-839 (maize); Hooykaas-Van Slogteren et al. (1984) Nature (London), 311:763-764; Bowen et al., U.S. Pat. No. 5,736,369 (cereals); Bytebier et al. (1987) Proc. Natl. Acad. Sci. USA, 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports, 9:415-418 and Kaeppler et al. (1992) Theor. Appl. Genet., 84:560-566 (whisker-mediated transformation); D'Halluin et al. (1992) Plant Cell, 4:1495-1505 (electroporation); Li et al. (1993) Plant Cell Reports, 12:250-255 and Christou and Ford (1995) Annals of Botany, 75:407-413 (rice); Ishida et al. (1996) Nature Biotechnology, 14:745-750; U.S. Pat. No. 5,731,179; U.S. Pat. No. 5,591,616; U.S. Pat. No. 5,641,664; and U.S. Pat. No. 5,981,840 (maize via Agrobacterium tumefaciens); the disclosures of which are herein incorporated by reference.

In planta Agrobacterium transformation is disclosed in the following: Bechtold, N., J. Ellis, G. Pelletier (1993) C. R., Acad Sci Paris Life Sci, 316:1194-1199; Bechtold, N., B. et al. (2000) Genetics, 155:1875-1887; Bechtold, N. and G. Pelletier (1998) Methods Mol Biol., 82:259-266; Chowrira, G. M., V. Akella, and P. F. Lurquin. (1995) Mol. Biotechnol., 3:17-23; Clough, S. J., and A. F. Bent. (1998) Plant J., 16:735-743; Desfeux, C., S. J. Clough, and A. F. Bent. (2000) Plant Physiol., 123: 895-904; Feldmann, K. A., and M. D. Marks. (1987) Mol. Gen. Genet., 208:1-9; Hu C.-Y., and L. Wang. (1999) In Vitro Cell Dev. Biol.-Plant 35:417-420; Katavic, V. G. W. Haughn, D. Reed, M. Martin, L. Kunst (1994) Mol. Gen. Genet., 245: 363-370; Liu, F., et al. (1998) Acta Hort 467:187-192; Mysore, K. S., C. T. Kumar, and S. B. Gelvin. (2000) Plant J., 21:9-16; Touraev, A., E. Stoger, V. Voronin, and E. Heberle-Bors. (1997) Plant J., 12:949-956; Trieu, A. T. et al. (2000) Plant J. 22:531-541; Ye, G. N. et al. (1999) Plant J., 19:249-257; Zhang, JU. et al. (2000) Chem Biol., 7:611-621. The disclosures of the above are herein incorporated by reference.

Various types of plant tissue can be used for transformation such as embryo cells, meristematic cells, leaf cells, or callus cells derived from embryo, leaf or meristematic cells. However, any transformation-competent cell or tissue can be used. Various methods for increasing transformation frequency may also be employed. Such methods are disclosed in WO 99/61619; WO 00/17364; WO 00/28058; WO 00/37645; U.S. Ser. No. 09/496,444; WO 00/50614; US01/44038; and WO 02/04649. The disclosures of the above are herein incorporated by reference.

Transformation of maize can follow a well-established bombardment transformation protocol used for introducing DNA into the scutellum of immature maize embryos (See, e.g., Tomes et al., Direct DNA Transfer into Intact Plant Cells Via Microprojectile Bombardment. pp. 197-213 in Plant Cell, Tissue and Organ Culture, Fundamental Methods. eds. O. L. Gamborg and G. C. Phillips. Springer-Verlag Berlin Heidelberg New York, 1995). Cells are transformed by culturing maize immature embryos (approximately 1-1.5 mm in length) onto medium containing N6 salts, Erikkson's vitamins, 0.69 g/l proline, 2 mg/l 2,4-D and 3% sucrose. After 4-5 days of incubation in the dark at 28° C., embryos are removed from the first medium and cultured onto similar medium containing 12% sucrose. Embryos are allowed to acclimate to this medium for 3 h prior to transformation. The scutellar surface of the immature embryos is targeted using particle bombardment. Embryos are transformed using the PDS-1000 Helium Gun from Bio-Rad at one shot per sample using 650PSI rupture disks. DNA delivered per shot averages at 0.1667 μg. Following bombardment, all embryos are maintained on standard maize culture medium (N6 salts, Erikkson's vitamins, 0.69 g/l proline, 2 mg/l 2,4-D, 3% sucrose) for 2-3 days and then transferred to N6-based medium containing a selective agent. Plates are maintained at 28° C. in the dark and are observed for colony recovery with transfers to fresh medium every two to three weeks. Recovered colonies and plants are scored based on the selectable or screenable phenotype imparted by the marker gene(s) introduced (i.e. herbicide resistance, fluorescence or anthocyanin production), and by molecular characterization via PCR and Southern analysis.

Transformation of maize can also be done using the Agrobacterium mediated DNA delivery method, as described by U.S. Pat. No. 5,981,840 with the following modifications. Agrobacteria are grown to the log phase in liquid minimal A medium containing 100 μM spectinomycin. Embryos are immersed in a log phase suspension of Agrobacteria adjusted to obtain an effective concentration of 5×10⁸ cfu/ml. Embryos are infected for 5 minutes and then co-cultured on culture medium containing acetosyringone for 7 days at 20° C. in the dark. After 7 days, the embryos are transferred to standard culture medium (MS salts with N6 macronutrients, 1 mg/L 2,4-D, 1 mg/L Dicamba, 20 g/L sucrose, 0.6 g/L glucose, 1 mg/L silver nitrate, and 100 mg/L carbenicillin) with a selective agent. Plates are maintained at 28° C. in the dark and are observed for colony recovery with transfers to fresh medium every two to three weeks. Recovered colonies and plants are scored based on the selectable or screenable phenotype imparted by the marker gene(s) introduced (i.e. herbicide resistance, fluorescence or anthocyanin production), and by molecular characterization via PCR and Southern analysis.

As used herein “regeneration” means the process of growing a plant from a plant cell (e.g., plant protoplast, callus or explant). It is contemplated that any cell from which a fertile plant may be regenerated is useful as a recipient cell. Callus may be initiated from tissue sources including, but not limited to, immature embryos, seedling apical meristems, microspores and the like. Those cells which are capable of proliferating as callus also are recipient cells for genetic transformation. Practical transformation methods and materials for making transgenic plants of this invention, e.g. various media and recipient target cells, transformation of immature embryos and subsequent regeneration of fertile transgenic plants are disclosed in U.S. Pat. No. 6,194,636, which is incorporated herein by reference.

As used herein a “transgenic” organism is one whose genome has been altered by the incorporation of foreign genetic material or additional copies of native genetic material, e.g. by transformation or recombination. The transgenic organism may be a plant, mammal, fungus, bacterium or virus. As used herein “transgenic plant” means a plant or progeny plant of any subsequent generation derived therefrom, wherein the DNA of the plant or progeny thereof contains an introduced exogenous DNA not originally present in a non-transgenic plant of the same strain. The transgenic plant may additionally contain sequences which are native to the plant being transformed, but wherein the exogenous DNA has been altered in order to alter the level or pattern of expression of the gene.

The present invention contemplates the use of polynucleotides which encode a protein or RNA product effective for imparting a desired characteristic to a plant, for example, increased yield. Such polynucleotides are assembled in recombinant DNA constructs using methods known to those of ordinary skill in the art. A useful technology for building DNA constructs and vectors for transformation is the GATEWAY® cloning technology (available from Invitrogen Life Technologies, Carlsbad, Calif.) which uses the site-specific recombinase LR cloning reaction of the Integrase/att system from bacterophage lambda vector construction, instead of restriction endonucleases and ligases. The LR cloning reaction is disclosed in U.S. Pat. Nos. 5,888,732 and 6, 277,608, U.S. Patent Application Publications 2001283529, 2001282319 and 20020007051, all of which are incorporated herein by reference. The GATEWAY® Cloning Technology Instruction Manual which is also supplied by Invitrogen also provides concise directions for routine cloning of any desired RNA into a vector comprising operable plant expression elements.

As used herein, “exogenous DNA” refers to DNA which does not naturally originate from the particular construct, cell or organism in which that DNA is found. Recombinant DNA constructs used for transforming plant cells will comprise exogenous DNA and usually other elements as discussed below. As used herein “transgene” means an exogenous DNA which has been incorporated into a host genome or is capable of autonomous replication in a host cell and is capable of causing the expression of one or more cellular products. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the exogenous DNA.

As used herein “gene” or “coding sequence” means a DNA sequence from which an RNA molecule is transcribed. The RNA may be an mRNA which encodes a protein product, an RNA which functions as an anti-sense molecule, or a structural RNA molecule such as a tRNA, rRNA, or snRNA, or other RNA. As used herein “expression” refers to the combination of intracellular processes, including transcription and translation, undergone by a DNA molecule, such as a structural gene to produce a polypeptide, or a non-structural gene to produce an RNA molecule.

As used herein “promoter” means a region of DNA sequence that is essential for the initiation of transcription of RNA from DNA; this region may also be referred to as a “5′ regulatory region.” Promoters are located upstream of DNA to be translated and have regions that act as binding sites for RNA polymerase and have regions that work with other factors to promote RNA transcription. More specifically, basal promoters in plants comprise canonical regions associated with the initiation of transcription, such as CAAT and TATA boxes. The TATA box element is usually located approximately 20 to 35 nucleotides upstream of the site of initiation of transcription. The CAAT box element is usually located approximately 40 to 200 nucleotides upstream of the start site of transcription. The location of these basal promoter elements result in the synthesis of an RNA transcript comprising some number of nucleotides upstream of the translational ATG start site. The region of RNA upstream of the ATG is commonly referred to as a 5′ untranslated region or 5′ UTR. It is possible to use standard molecular biology techniques to make combinations of basal promoters, that is regions comprising sequences from the CAAT box to the translational start site, with other upstream promoter elements to enhance or otherwise alter promoter activity or specificity.

As is well known in the art, recombinant DNA constructs typically also comprise other regulatory elements in addition to a promoter, such as but not limited to 3′ untranslated regions (such as polyadenylation sites), transit or signal peptides and marker genes elements. For instance, see U.S. Pat. No. 6,437,217 which discloses a maize RS81 promoter, U.S. Pat. No. 5,641,876 which discloses a rice actin promoter, U.S. Pat. No. 6,426,446 which discloses a maize RS324 promoter, U.S. Pat. No. 6,429,362 which discloses a maize PR-1 promoter, U.S. Pat. No. 6,232,526 which discloses a maize A3 promoter, U.S. Pat. No. 6,177,611 which discloses constitutive maize promoters, U.S. Pat. No. 6,433,252 which discloses a maize L3 oleosin promoter, U.S. Pat. No. 6,429,357 which discloses a rice actin 2 promoter and intron, U.S. Pat. No. 5,837,848 which discloses a root specific promoter, U.S. Pat. No. 6,084,089 which discloses cold inducible promoters, U.S. Pat. No. 6,294,714 which discloses light inducible promoters, U.S. Pat. No. 6,140,078 which discloses salt inducible promoters, U.S. Pat. No. 6,252,138 which discloses pathogen inducible promoters, U.S. Pat. No. 6,175,060 which discloses phosphorus deficiency inducible promoters, U.S. Patent Application Publication 2002/0192813A1 which discloses 5′, 3′ and intron elements useful in the design of effective plant expression vectors, U.S. patent application Ser. No. 09/078,972 which discloses a coixin promoter, and U.S. patent application Ser. No. 09/757,089 which discloses a maize chloroplast aldolase promoter, all of which are incorporated herein by reference.

Cells may be tested further to confirm stable integration of the exogenous DNA. Useful selective marker genes include those conferring resistance to antibiotics such as kanamycin (nptII), hygromycin B (aph IV) and gentamycin (aac3 and aacC4) or resistance to herbicides such as glufosinate (bar or pat) and glyphosate (EPSPS; CP4). Examples of such selectable markers are illustrated in U.S. Pat. Nos. 5,550,318; 5,633,435; 5,780,708 and 6,118,047, all of which are incorporated herein by reference. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

An important advantage of the present invention is that it provides methods and compositions for the efficient transformation of selected genes and regeneration of plants with desired agronomic traits. In this way, yield and other agronomic testing schemes can be carried out earlier in the commercialization process.

The choice of a selected gene for expression in a plant host cell in accordance with the invention will depend on the purpose of the transformation. One of the major purposes of transformation of crop plants is to add commercially desirable, agronomically important or end-product traits to the plant. Such traits include, but are not limited to, herbicide resistance or tolerance, insect resistance or tolerance, disease resistance or tolerance (viral, bacterial, fungal, nematode), stress tolerance and/or resistance, as exemplified by resistance or tolerance to drought, heat, chilling, freezing, excessive moisture, salt stress and oxidative stress, increased yield, food or feed content and value, physical appearance, male sterility, drydown, standability, prolificacy, starch quantity and quality, oil quantity and quality, protein quality and quantity, amino acid composition, and the like.

In certain embodiments of the invention, transformation of a recipient cell may be carried out with more than one exogenous (selected) gene. As used herein, an “exogenous coding region” or “selected coding region” is a coding region not normally found in the host genome in an identical context. By this, it is meant that the coding region may be isolated from a different species than that of the host genome, or alternatively, isolated from the host genome, but is operably linked to one or more regulatory regions which differ from those found in the unaltered, native gene. Two or more exogenous coding regions also can be supplied in a single transformation event using either distinct transgene-encoding vectors, or using a single vector incorporating two or more coding sequences. Any two or more transgenes of any description, such as those conferring herbicide, insect, disease (viral, bacterial, fungal, nematode) or drought resistance, male sterility, drydown, standability, prolificacy, starch properties, oil quantity and quality, or those increasing yield or nutritional quality may be employed as desired.

In addition to direct transformation of a particular plant genotype, such as an elite line with enhanced transformability, with a construct prepared according to the current invention, transgenic plants may be made by crossing a plant having a construct of the invention to a second plant lacking the construct. For example, a selected coding region can be introduced into a particular plant variety by crossing, without the need for ever directly transforming a plant of that given variety. Therefore, the current invention not only encompasses a plant directly regenerated from cells which have been transformed in accordance with the current invention, but also the progeny of such plants. As used herein the term “progeny” denotes the offspring of any generation of a parent plant prepared in accordance with the instant invention, wherein the progeny comprises a construct prepared in accordance with the invention. “Crossing” a plant to provide a plant line having one or more added transgenes relative to a starting plant line, as disclosed herein, is defined as the techniques that result in a transgene of the invention being introduced into a plant line by crossing a starting line with a donor plant line that comprises a transgene of the invention. To achieve this one could, for example, perform the following steps:

(a) plant seeds of the first (starting line) and second (donor plant line that comprises a transgene of the invention) parent plants;

(b) grow the seeds of the first and second parent plants into plants that bear flowers;

(c) pollinate a flower from the first parent plant with pollen from the second parent plant; and

(d) harvest seeds produced on the parent plant bearing the fertilized flower.

Backcrossing is herein defined as the process including the steps of:

(a) crossing a plant of a first genotype containing a desired gene, DNA sequence or element to a plant of a second genotype lacking said desired gene, DNA sequence or element;

(b) selecting one or more progeny plant containing the desired gene, DNA sequence or element;

(c) crossing the progeny plant to a plant of the second genotype; and

(d) repeating steps (b) and (c) for the purpose of transferring said desired gene, DNA sequence or element from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking said desired DNA sequence may be referred to as an unconverted genotype, line, inbred, or hybrid.

The following examples are included to illustrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

All publications and patent applications mentioned in this specification are herein incorporated by reference to the same extent as if each individual publication or patent application was specifically and individually indicated to be incorporated by reference.

The following examples are offered by way of illustration and not by way of limitation.

### EXAMPLE 1

### Transformability Analysis of the Doubled Haploid Lines Derived from Hi-II

Hi-II is a corn hybrid that is easy to culture and regenerate (Armstrong et al. 1991 and 1992). It has been broadly used for genetic transformation via bombardment (Gordon-Kamm et al. 1990; Songstad et al. 1996; and O'kennedy et al. 1998) and Agrobacterium (Zhao et al. 1998 and 2001; Frame et al. 2002).

Doubled haploid plants were derived by pollinating Hi-II plants by a haploid inducer line, RWS. These doubled haploid plants contain two sets of homozygous chromosomes derived from only the Hi-II parent. The male parent, RWS, did not make any chromosomal contribution to the doubled haploid plants. Because Hi-II is a hybrid derived from two different parents, parent A and parent B, the doubled haploid plants derived from Hi-II are the results of gene recombination and segregation during meiosis of the female parent. Individual doubled haploid plants represent a unique recombination and they are each genetically different from one another. These doubled haploid plants provide good genetic material for the analysis used to determine the genetic basis of transformability.

Each unique doubled haploid plant was self-pollinated to produce double haploid seeds. The doubled haploid seeds obtained from one selfed plant form a homozygous line. Through this process, twenty double haploid lines are produced from the Hi-II plants which are considered F1 plants. The seeds of the twenty double haploid lines were planted and the immature embryos from each of the twenty double haploid lines were evaluated for transformability.

The method of Agrobacterium mediated maize transformation (Zhao et al. 2001) is used for evaluation of the transformability of these lines. The immature embryos (9-12 days after pollination) isolated from these double haploid lines are infected with Agrobacterium that harbored a super-binary vector and the T-DNA contains a selectable marker gene and a visible marker gene. The evaluation includes 1) the type of callus (type I or type II or mix of type I and II etc.); 2) level of T-DNA delivered into embryos (based on level of transient expression of the visible marker gene in the embryos following Agrobacterium infection); 3) frequency of stable transformation (based on the resistance of the callus tissue to selective agent and expression of the visible marker gene in the same callus tissue); 4) frequency of plant regeneration (based on the expression of both selectable marker gene and visible marker gene in the regenerated plants to confirm the frequency stable transformed plants regenerated from the putative transformed callus tissues). The results of the evaluation are listed in Table 1. For each category, 4 scales are used to measure the results. Callus Types: 1=high quality of type II callus, 2=low quality of type II callus with non-embryogenic tissues, 3=mix of type I and type II callus, 4=type I callus, 5=low quality of type I, 6=no callus response. Frequency of stable transformation (%): 1=15% or higher, 2=5-14%, 3=1-4%, 4=0%. Plant Regeneration Frequency (%): 1=80% or higher, 2=50-79%, 3=1-49%, 4=0%.

TABLE 1
Transformability analysis of Doubled Haploid Lines Derived from Hi-II

| | | | Plant Regeneration |
|---|---|---|---|
| Line No. | Callus Type | Stable Transformation % | % |
| 1 | 1 | 1 | 1 |
| 2 | 1 | 1 | 1 |
| 3 | 1 | 4 | NA |
| 4 | 1 | 3 | 4 |
| 5 | 1 | 3 | 4 |
| 6 | 1 | 4 | NA |
| 7 | 1 | 3 | 4 |
| 8 | 1 | 4 | NA |
| 9 | 1 | 2 | 1 |
| 10 | 1 | 3 | 1 |
| 11 | 1 | 2 | 1 |
| 12 | 1 | 1 | 1 |
| 13 | 1 | 1 | 1 |
| 14 | 1 | 1 | 1 |
| 15 | 1 | 1 | 2 |
| 16 | 1 | 3 | 4 |
| 17 | 1 | 1 | 1 |
| 18 | 1 | 2 | 1 |
| 19 | 1 | 4 | NA |
| 20 | 1 | 3 | 4 |

Lines 1, 2, 12, 13, and 17 showed high level T-DNA delivery, high frequency of callus transformation and high frequency of plant regeneration. These five lines are highly transformable. Line 14 showed intermediated T-DNA delivery and high frequency of stable transformation and plant regeneration and it is still considered a highly transformable line. Lines 3, 6, 8 showed high T-DNA deliveries, but no stable transformed callus was recovered. Because these lines did not produce stable transformed callus, plant regeneration could not be evaluated.

### EXAMPLE 2

Identification of markers associated with transformability though analysis of doubled haploid lines from Hi-II. These 20 doubled haploid lines derived from Hi-II were used to identify the markers associated with transformability.

Different types of molecular markers could be employed to map genes that significantly affect the transformability. In this study, Simple Sequence Repeat (or SSR or microsatellite) markers were employed. SSR markers are PCR based DNA markers. The sizes of the PCR products as visualized after electrophoresis are used as differentiating characteristics of the individual for the locus under study. A number of publicly available SSR molecular markers are available to carry out studies like this and can be found on the world wide web at agron.missouri.edussr.html//mapfiles.

Only the markers that discriminate the parents of the population are useful since those will track one of the alternate alleles possible in a segregating population. The parents of the Hi-II, Parent A and Parent B, were screened using the SSR markers. The polymorphic markers were then selected to use in the population. While selecting the markers, the genome coverage, quality of the markers (robustness) and the information content (as measured by PIC) were considered.

Marker-Trait Association Analysis Methods and Results

The statistical associations of SSR markers with transformability traits are reported in Table 2A-2B, and Table 3A-3B. The column 1, 2, 3 of each table give the names of SSR markers, their chromosome IDs, and their positions on a chromosome in map distance (centiMorgan, or cM) based on the IBM Genetic Linkage Map. The sample size given in column 4 of Table 2A and Table 3A are the number of DH lines actually used in trait-marker association tests.

The statistical association between a trait and marker is measured using a general linear statistical model implemented in SAS Version 9.0 (SAS Institute, Cary, N.C.). The model measures the proportion of total trait phenotypic variation that can be attributed to the marker allele state change. A larger proportion indicates stronger association between the trait value and the marker allele state. F test is used to measure statistical significance (column 5). An F test result that is significant at P value less than 10% (P<0.1) is taken as the evidence of significant association. Pair-wise association between each of the total 239 markers and a trait is tested by F test and only the markers that show significant association (column 6) are reported in Table 2A and Table 3A.

Table 2B and 3B show the allele state (column 5), the number of DH lines that have the allele state (sample size, column 6) and the mean (column 7) and the standard deviation (SD) (column 8) of their trait values. The Trait Mean and Trait SD (column 7, 8) are computed using the all the DH lines that have the same allele state. Large difference in mean trait values among the DH lines of different allele state are evident for all the markers we reported. Our association tests show that one SSR marker, MARKER D on chromosome 5 at map position 91 cM is associated with Stable Transformation Percentage (Table 2A, 2B) and seven SSR markers located on four different chromosomes are associated with plant regeneration (Table 3A and 3B).

TABLE 2A
Markers Significantly Associated with Transformation Percentage
in Hi-II Double Haploid lines

| | | Marker | Sample | F | P |
|---|---|---|---|---|---|
| Chromosome | Position | Name | Size | Value | Value |
| 5 | 91 | MARKER D | 16 | 3.15 | 0.10 |

TABLE 2B
Allele Types and Allele Phenotype Means from Table 2A.

| | | Marker | | Sample | Trait | Trait |
|---|---|---|---|---|---|---|
| Chromosome | Position | Name | Allele | Size | Mean | SD |
| 5 | 91 | MARKER D | A | 2 | 1 | 0.00 |
| 5 | 91 | MARKER D | B | 14 | 2.5 | 1.16 |

TABLE 3A
Markers Significantly Associated with Plant Regeneration
in Hi-II Double Haploid Lines

| | | Marker | Sample | F | P |
|---|---|---|---|---|---|
| Chromosome | Position | Name | Size | Value | Value |
| 1 | 30 | BNLG1014 | 15 | 4.42 | 0.06 |
| 1 | 213 | UMC1254 | 14 | 7.75 | 0.02 |
| 5 | 203 | UMC2013 | 14 | 3.43 | 0.09 |
| 5 | 215 | UMC1792 | 8 | 9.00 | 0.02 |
| 7 | 0 | MARKER J | 13 | 5.29 | 0.04 |
| 7 | 151 | UMC2133 | 14 | 3.57 | 0.08 |
| 7 | 161 | UMC1708 | 12 | 4.05 | 0.07 |
| 9 | 79 | UMC2087 | 11 | 3.41 | 0.10 |

TABLE 3B
Allele Types and Allele Phenotype Means from Table 3A.

| | | Marker | | Sample | Trait | Trait |
|---|---|---|---|---|---|---|
| Chromosome | Position | Name | Allele | Size | Mean | SD |
| 1 | 30 | BNLG1014 | A | 5 | 2.80 | 1.64 |
| 1 | 30 | BNLG1014 | F | 10 | 1.40 | 0.97 |
| 1 | 213 | UMC1254 | D | 4 | 3.25 | 1.50 |
| 1 | 213 | UMC1254 | E | 10 | 1.40 | 0.97 |
| 5 | 203 | UMC2013 | D | 10 | 2.50 | 1.58 |
| 5 | 203 | UMC2013 | E | 4 | 1.00 | 0.00 |
| 5 | 215 | UMC1792 | A | 3 | 1.00 | 0.00 |
| 5 | 215 | UMC1792 | B | 5 | 3.40 | 1.34 |
| 7 | 0 | MARKER J | C | 7 | 2.43 | 1.51 |
| 7 | 0 | MARKER J | D | 6 | 1.00 | 0.00 |
| 7 | 151 | UMC2133 | B | 6 | 2.67 | 1.51 |
| 7 | 151 | UMC2133 | C | 8 | 1.38 | 1.06 |
| 7 | 161 | UMC1708 | A | 9 | 2.78 | 1.48 |
| 7 | 161 | UMC1708 | C | 3 | 1.00 | 0.00 |
| 9 | 79 | UMC2087 | A | 4 | 1.00 | 0.00 |
| 9 | 79 | UMC2087 | B | 7 | 2.43 | 1.51 |

### EXAMPLE 3

### Transformability Analysis of the Doubled Haploid Lines Derived from Hi-II x Gaspe Flint

Hi-II is used as the female parent and Gaspe Flint, a near-inbred line, is used as the male parent to make the F1 hybrid. The plants of this hybrid are pollinated with haploid inducer, RWS, to generate haploid immature embryos. These haploid immature embryos are cultured on tissue culture medium to produce callus. The callus tissues are treated with chromosomal doubling agent, such as colchicine or pronamide, to produce doubled haploid callus tissues. These doubled haploid tissues are used to generate doubled haploid plants. The doubled haploid plants are self-pollinated to produce doubled haploid seeds. The seeds derived from each single haploid embryo make a doubled haploid line.

Fifty of these doubled haploid lines are evaluated for transformability. The method of Agrobacterium mediated maize transformation (Zhao et al. 2001) is used for evaluation of the transformability of these lines. The immature embryos (9-12 days after pollination) isolated from these double haploid lines are infected with Agrobacterium that harbored a super-binary vector and the T-DNA contains a selectable marker gene and other genes. The evaluation includes 1) the type of callus (type I or type II or mix of type I and II etc.); 2) frequency of stable transformation (based on the resistance of the callus tissue to selective agent); 3) frequency of plant regeneration (based on the expression of selectable marker gene in the regenerated plants to confirm the frequency stable transformed plants regenerated from the putative transformed callus tissues). The results of the evaluation are listed in Table 4. For each category, 4 scales are used to measure the results. Callus Types: 1=high quality of type II callus, 2=low quality of type II callus with non-embryogenic tissues, 3=mix of type I and type II callus, 4=high quality of type I callus, 5=low quality of type I, 6=no callus response. Stable Transformation Frequency (%): 1=15% or higher, 2=5-14%, 3=1-4%, 4=0%. Plant Regeneration Frequency (%): 1=80% or higher, 2=50-79%, 3=1-49%, 4=0%.

TABLE 4
Transformability analysis of Doubled Haploid Lines Derived from
Hi-II × Gaspe Flint

| | | | Plant Regeneration |
|---|---|---|---|
| Line No. | Callus Type | Stable Transformation % | % |
| 1 | 1 | 1 | 1 |
| 2 | 1 | 1 | 1 |
| 3 | 1 | 1 | 2 |
| 4 | 2 | 1 | 1 |
| 5 | 1 | 1 | 1 |
| 6 | 5 | 4 | 4 |
| 7 | 1 | 2 | 1 |
| 8 | 2 | 2 | |
| 9 | 1 | 3 | 1 |
| 10 | 1 | 1 | 1 |
| 11 | 1 | 1 | 1 |
| 12 | 3 | 2 | |
| 13 | 1 | 1 | |
| 14 | 3 | 1 | 1 |
| 15 | 5 | 1 | |
| 16 | 5 | 2 | |
| 17 | 3 | 1 | 1 |
| 18 | 5 | 1 | |
| 19 | 1 | 1 | 1 |
| 20 | 5 | 4 | |
| 21 | 2 | 1 | |
| 22 | 2 | 1 | 2 |
| 23 | 2 | 1 | 2 |
| 24 | 2 | 2 | 2 |
| 25 | 2 | 1 | 1 |
| 26 | 2 | 1 | |
| 27 | 2 | 2 | |
| 28 | 2 | 1 | |
| 29 | 5 | 2 | |
| 30 | 2 | 3 | |
| 31 | 3 | 2 | |
| 32 | 5 | 2 | |
| 33 | 2 | 3 | |
| 34 | 1 | 3 | |
| 35 | 1 | 3 | |
| 36 | 3 | 2 | 1 |
| 37 | 3 | 1 | 1 |
| 38 | 2 | 2 | 1 |
| 39 | 2 | 3 | |
| 40 | 3 | 1 | 1 |
| 41 | 5 | 3 | |
| 42 | 1 | 1 | 1 |
| 43 | 1 | 1 | 1 |
| 44 | 5 | 2 | |
| 45 | 1 | 3 | |
| 46 | 2 | 1 | 1 |
| 47 | 3 | 2 | |
| 48 | 2 | 1 | |
| 49 | 5 | 1 | |

### EXAMPLE 4

### Identification of Markers Associated with Transformability Thought Analysis of Doubled Haploid Lines from Hi-II x Gaspe Flint

SSR markers were used to identify the associated regions in the genome that increase the transformability. The parents, Hi-II and Gaspe Flint, are evaluated with all the SSR production markers and the polymorphic markers were identified. A set of marker that are evenly distributed through out the genome are selected which also are robust and have high PIC (polymorphic Information Content) value. These markers were then assayed with the DNA extracted from the leaf material of the doubled haploid population derived from the Hi-II X Gaspe Flint cross. The PCR products are electrophoresed to find the characteristic base pair inherited from either parent.

Market-Trait Association Analysis Methods and Results

The statistical associations of SSR markers with transformability traits are reported in Table 5A-5B, Table 6A-6B, and Table 7A-7B. The column 1, 2, 3 of each table give the names of SSR markers, their chromosome IDs, and their positions on a chromosome in map distance (centiMorgan, or cM). The genetic map and SSR marker set used for association analysis in this example is the same as the Example 2. The sample size given in column 4 of Table 5A, 6A, and 7A are the number of DH lines actually used in trait-marker association tests.

The statistical association between a trait and marker is measured using the same statistical procedure for Example 2. The method measures the proportion of total trait phenotypic variation that can be attributed to marker allele state change. A larger proportion indicates stronger association between the trait value and the marker allele state. F test is used to measure statistical significance (column 5). A F test result that is significant at P value less than 10% (P<0.1) is taken as the evidence of significant statistical association. Pair-wise association between each of the total 239 markers and a trait is tested by F test and only the markers that show significant association (column 6) are reported in Table 5A, 6A, and 7A.

Table 5B, 6B, and 7B show the allele state (column 5), the number of DH lines that have the allele state (sample size, column 6) and the mean (column 7) and the standard deviation (SD) (column 8) of their trait values. The Trait Mean and Trait SD (column 7, 8) are computed using the all the DH lines that have the same allele state. Large difference in mean trait values among the DH lines of different allele state are evident for all the markers we reported.

Our association tests identify 17 SSR markers that are associated with Callus Type (Table 5A, 5B), 34 SSR markers that are associated with Callus Transformation Percentage (Table 6A, 6B) and 17 SSR markers that are associated with plant regeneration (Table 7A and 7B) in Hi-II x Gaspe Flint population.

TABLE 5A
Markers Significantly Associated with Callus Type
in Hi-II × Gaspe Flint Double Haploid Lines

| | | Marker | Sample | F | P |
|---|---|---|---|---|---|
| Chromosome | Position | Name | Size | Value | Value |
| 1 | 213 | UMC1254 | 43 | 3.73 | 0.03 |
| 1 | 330 | UMC1774 | 36 | 5.67 | 0.02 |
| 1 | 399 | UMC1797 | 44 | 3.01 | 0.06 |
| 2 | 29 | UMC1265 | 35 | 3.33 | 0.08 |
| 3 | 1 | PHI453121 | 41 | 3.27 | 0.08 |
| 4 | 142 | MARKER E | 45 | 8.40 | 0.01 |
| 4 | 174 | UMC2041 | 46 | 9.43 | 0.00 |
| 4 | 195 | MARKER G | 31 | 2.42 | 0.09 |
| 5 | 42 | UMC1365 | 37 | 3.38 | 0.05 |
| 5 | 70 | MARKER F | 41 | 3.41 | 0.07 |
| 5 | 75 | UMC2035 | 48 | 3.39 | 0.07 |
| 5 | 78 | UMC2294 | 44 | 3.73 | 0.06 |
| 7 | 66 | UMC1339 | 40 | 5.55 | 0.01 |
| 7 | 68 | UMC1433 | 31 | 3.27 | 0.08 |
| 8 | 146 | UMC1287 | 46 | 3.15 | 0.08 |
| 8 | 165 | UMC1607 | 41 | 3.36 | 0.07 |
| 8 | 184 | BNLG1828 | 39 | 3.59 | 0.07 |

TABLE 5B
Allele Types and Allele Phenotype Means from Table 5A.

| | | Marker | | Sample | Trait | Trait |
|---|---|---|---|---|---|---|
| Chromosome | Position | Name | Allele | Size | Mean | SD |
| 1 | 213 | UMC1254 | C | 27 | 2.19 | 1.59 |
| 1 | 213 | UMC1254 | D | 1 | 6.00 | 0.00 |
| 1 | 213 | UMC1254 | E | 15 | 2.67 | 1.05 |
| 1 | 330 | UMC1774 | A | 19 | 3.00 | 1.80 |
| 1 | 330 | UMC1774 | B | 17 | 1.82 | 1.01 |
| 1 | 399 | UMC1797 | A | 7 | 1.86 | 0.69 |
| 1 | 399 | UMC1797 | G | 14 | 1.93 | 1.21 |
| 1 | 399 | UMC1797 | L | 23 | 3.00 | 1.76 |
| 2 | 29 | UMC1265 | F | 24 | 2.42 | 1.47 |
| 2 | 29 | UMC1265 | G | 11 | 3.45 | 1.75 |
| 3 | 1 | PHI453121 | A | 22 | 2.77 | 1.74 |
| 3 | 1 | PHI453121 | C | 19 | 1.95 | 1.03 |
| 4 | 142 | MARKER E | A | 24 | 1.92 | 1.14 |
| 4 | 142 | MARKER E | C | 21 | 3.19 | 1.78 |
| 4 | 174 | UMC2041 | B | 25 | 2.00 | 1.15 |
| 4 | 174 | UMC2041 | C | 21 | 3.33 | 1.77 |
| 4 | 195 | MARKER G | C | 15 | 2.87 | 1.96 |
| 4 | 195 | MARKER G | D | 1 | 1.00 | 0.00 |
| 4 | 195 | MARKER G | L | 14 | 2.14 | 1.03 |
| 4 | 195 | MARKER G | R | 1 | 6.00 | 0.00 |
| 5 | 42 | UMC1365 | A | 9 | 2.44 | 1.59 |
| 5 | 42 | UMC1365 | B | 18 | 3.11 | 1.71 |
| 5 | 42 | UMC1365 | C | 10 | 1.60 | 0.70 |
| 5 | 70 | MARKER F | B | 13 | 3.38 | 1.80 |
| 5 | 70 | MARKER F | C | 28 | 2.39 | 1.50 |
| 5 | 75 | UMC2035 | A | 18 | 3.00 | 1.68 |
| 5 | 75 | UMC2035 | D | 30 | 2.17 | 1.42 |
| 5 | 78 | UMC2294 | A | 29 | 2.17 | 1.44 |
| 5 | 78 | UMC2294 | B | 15 | 3.07 | 1.49 |
| 7 | 66 | UMC1339 | B | 4 | 3.00 | 1.41 |
| 7 | 66 | UMC1339 | C | 13 | 1.54 | 0.66 |
| 7 | 66 | UMC1339 | D | 23 | 3.09 | 1.62 |
| 7 | 68 | UMC1433 | A | 12 | 2.83 | 1.64 |
| 7 | 68 | UMC1433 | B | 19 | 1.89 | 1.24 |
| 8 | 146 | UMC1287 | D | 26 | 2.08 | 1.16 |
| 8 | 146 | UMC1287 | G | 20 | 2.85 | 1.79 |
| 8 | 165 | UMC1607 | B | 19 | 2.05 | 1.22 |
| 8 | 165 | UMC1607 | C | 22 | 2.91 | 1.69 |
| 8 | 184 | BNLG1828 | B | 18 | 1.89 | 0.76 |
| 8 | 184 | BNLG1828 | F | 21 | 2.71 | 1.71 |

TABLE 6A
Markers Significantly Associated with Transformation
Percentage in Hi-II × Gaspe Flint Double Haploid Lines

| | | Marker | Sample | F | P |
|---|---|---|---|---|---|
| Chromosome | Position | Name | Size | Value | Value |
| 1 | 88 | UMC1701 | 69 | 4.97 | 0.03 |
| 1 | 213 | UMC1254 | 73 | 2.89 | 0.06 |
| 1 | 241 | UMC1119 | 71 | 3.15 | 0.08 |
| 1 | 320 | BNLG1720 | 65 | 3.09 | 0.03 |
| 2 | 29 | UMC1265 | 61 | 6.78 | 0.01 |
| 2 | 214 | BNLG1520 | 71 | 2.39 | 0.10 |
| 3 | 18 | UMC1458 | 62 | 6.91 | 0.01 |
| 3 | 107 | UMC1174 | 35 | 5.12 | 0.03 |
| 3 | 110 | UMC1167 | 75 | 4.69 | 0.03 |
| 4 | 90 | MARKER B | 74 | 5.03 | 0.01 |
| 4 | 97 | UMC1662 | 59 | 5.53 | 0.02 |
| 4 | 101 | UMC1895 | 74 | 4.52 | 0.04 |
| 4 | 106 | UMC1142 | 51 | 4.95 | 0.03 |
| 4 | 142 | MARKER E | 74 | 3.09 | 0.08 |
| 5 | 41 | UMC2036 | 58 | 5.89 | 0.02 |
| 5 | 42 | UMC1365 | 60 | 4.08 | 0.02 |
| 5 | 203 | UMC2013 | 75 | 3.49 | 0.04 |
| 5 | 215 | UMC1792 | 70 | 3.03 | 0.05 |
| 5 | 220 | UMC1225 | 76 | 2.93 | 0.06 |
| 5 | 231 | BNLG386 | 70 | 3.06 | 0.08 |
| 5 | 232 | UMC1153 | 72 | 4.83 | 0.03 |
| 6 | 43 | UMC1229 | 75 | 6.71 | 0.01 |
| 6 | 51 | UMC1195 | 73 | 7.76 | 0.01 |
| 6 | 108 | UMC1114 | 56 | 2.51 | 0.09 |
| 6 | 194 | UMC2059 | 62 | 4.25 | 0.04 |
| 7 | 140 | MARKER H | 73 | 3.15 | 0.05 |
| 7 | 151 | UMC2133 | 74 | 4.19 | 0.02 |
| 8 | 77 | UMC1910 | 53 | 4.29 | 0.04 |
| 9 | 33 | UMC1170 | 72 | 3.54 | 0.03 |
| 9 | 125 | UMC2341 | 45 | 2.82 | 0.07 |
| 9 | 153 | UMC2346 | 76 | 3.95 | 0.05 |
| 9 | 192 | BNGL619 | 74 | 3.46 | 0.04 |
| 9 | 196 | UMC2131 | 71 | 5.18 | 0.03 |
| 10 | 9 | PHI041 | 56 | 6.55 | 0.01 |

TABLE 6B
Allele Types and Allele Phenotype Means from Table 6A.

| | | Marker | | Sample | Trait | Trait |
|---|---|---|---|---|---|---|
| Chromosome | Position | Name | Allele | Size | Mean | SD |
| 1 | 88 | UMC1701 | A | 27 | 2.63 | 1.18 |
| 1 | 88 | UMC1701 | D | 42 | 2.02 | 1.05 |
| 1 | 213 | UMC1254 | C | 45 | 2.13 | 1.01 |
| 1 | 213 | UMC1254 | D | 3 | 3.67 | 0.58 |
| 1 | 213 | UMC1254 | E | 25 | 2.36 | 1.25 |
| 1 | 241 | UMC1119 | B | 33 | 2.58 | 1.15 |
| 1 | 241 | UMC1119 | C | 38 | 2.11 | 1.09 |
| 1 | 320 | BNLG1720 | A | 17 | 2.88 | 1.05 |
| 1 | 320 | BLNG1720 | B | 14 | 1.79 | 1.12 |
| 1 | 320 | BLNG1720 | C | 33 | 2.30 | 1.10 |
| 1 | 320 | BLNG1720 | D | 1 | 1.00 | 0.00 |
| 2 | 29 | UMC1265 | F | 32 | 1.91 | 1.20 |
| 2 | 29 | UMC1265 | G | 29 | 2.62 | 0.90 |
| 2 | 214 | BNLG1520 | B | 14 | 2.71 | 1.14 |
| 2 | 214 | BNLG1520 | C | 32 | 2.31 | 1.15 |
| 2 | 214 | BNLG1520 | D | 25 | 1.92 | 1.04 |
| 3 | 18 | UMC1458 | C | 26 | 1.73 | 1.00 |
| 3 | 18 | UMC1458 | F | 36 | 2.47 | 1.16 |
| 3 | 107 | UMC1174 | C | 26 | 2.62 | 1.27 |
| 3 | 107 | UMC1174 | D | 9 | 1.56 | 1.01 |
| 3 | 110 | UMC1167 | C | 45 | 2.47 | 1.18 |
| 3 | 110 | UMC1167 | E | 30 | 1.90 | 0.99 |
| 4 | 90 | MARKER B | B | 39 | 1.95 | 1.05 |
| 4 | 90 | MARKER B | C | 14 | 3.00 | 0.96 |
| 4 | 90 | MARKER B | E | 21 | 2.38 | 1.20 |
| 4 | 97 | UMC1662 | A | 30 | 2.63 | 1.07 |
| 4 | 97 | UMC1662 | C | 29 | 2.00 | 1.00 |
| 4 | 101 | UMC1895 | A | 37 | 2.57 | 1.09 |
| 4 | 101 | UMC1895 | B | 37 | 2.03 | 1.09 |
| 4 | 106 | UMC1142 | A | 28 | 2.00 | 1.05 |
| 4 | 106 | UMC1142 | B | 23 | 2.65 | 1.03 |
| 4 | 142 | MARKER E | A | 33 | 2.03 | 1.16 |
| 4 | 142 | MARKER E | C | 41 | 2.49 | 1.08 |
| 5 | 41 | UMC2036 | A | 23 | 2.61 | 1.12 |
| 5 | 41 | UMC2036 | B | 35 | 1.89 | 1.11 |
| 5 | 42 | UMC1365 | A | 11 | 1.55 | 0.93 |
| 5 | 42 | UMC1365 | B | 31 | 2.55 | 1.12 |
| 5 | 42 | UMC1365 | C | 18 | 1.94 | 1.11 |
| 5 | 203 | UMC2013 | B | 34 | 2.41 | 1.16 |
| 5 | 203 | UMC2013 | D | 26 | 2.46 | 1.17 |
| 5 | 203 | UMC2013 | E | 15 | 1.60 | 0.74 |
| 5 | 215 | UMC1792 | A | 15 | 1.80 | 0.86 |
| 5 | 215 | UMC1792 | B | 24 | 2.13 | 1.15 |
| 5 | 215 | UMC1792 | D | 31 | 2.61 | 1.17 |
| 5 | 220 | UMC1225 | A | 33 | 2.61 | 1.14 |
| 5 | 220 | UMC1225 | B | 15 | 1.80 | 0.86 |
| 5 | 220 | UMC1225 | C | 28 | 2.18 | 1.19 |
| 5 | 231 | BNLG386 | A | 28 | 2.54 | 1.23 |
| 5 | 231 | BNLG386 | B | 42 | 2.05 | 1.08 |
| 5 | 232 | UMC1153 | A | 30 | 2.60 | 1.13 |
| 5 | 232 | UMC1153 | C | 42 | 2.02 | 1.07 |
| 6 | 43 | UMC1229 | B | 33 | 2.67 | 1.19 |
| 6 | 43 | UMC1229 | H | 42 | 2.00 | 1.04 |
| 6 | 51 | UMC1195 | B | 29 | 2.69 | 1.17 |
| 6 | 51 | UMC1195 | D | 44 | 1.98 | 1.00 |
| 6 | 108 | UMC1114 | A | 4 | 1.00 | 0.00 |
| 6 | 108 | UMC1114 | C | 24 | 2.13 | 1.03 |
| 6 | 108 | UMC1114 | D | 28 | 2.25 | 1.11 |
| 6 | 194 | UMC2059 | B | 13 | 1.69 | 0.75 |
| 6 | 194 | UMC2059 | C | 49 | 2.37 | 1.11 |
| 7 | 140 | MARKER H | A | 27 | 2.63 | 1.08 |
| 7 | 140 | MARKER H | C | 10 | 1.60 | 0.70 |
| 7 | 140 | MARKER H | E | 36 | 2.31 | 1.21 |
| 7 | 151 | UMC2133 | A | 38 | 2.21 | 1.17 |
| 7 | 151 | UMC2133 | B | 17 | 2.82 | 1.07 |
| 7 | 151 | UMC2133 | C | 19 | 1.79 | 0.85 |
| 8 | 77 | UMC1910 | B | 44 | 2.09 | 1.07 |
| 8 | 77 | UMC1910 | E | 9 | 2.89 | 0.93 |
| 9 | 33 | UMC1170 | A | 34 | 2.12 | 1.12 |
| 9 | 33 | UMC1170 | F | 3 | 1.00 | 0.00 |
| 9 | 33 | UMC1170 | G | 35 | 2.54 | 1.07 |
| 9 | 125 | UMC2341 | A | 30 | 2.17 | 0.99 |
| 9 | 125 | UMC2341 | B | 1 | 1.00 | 0.00 |
| 9 | 125 | UMC2341 | C | 14 | 2.86 | 1.23 |
| 9 | 153 | UMC2346 | C | 32 | 1.94 | 0.91 |
| 9 | 153 | UMC2346 | D | 44 | 2.45 | 1.25 |
| 9 | 192 | BNGL619 | N | 31 | 2.00 | 1.00 |
| 9 | 192 | BNGL619 | T | 2 | 1.00 | 0.00 |
| 9 | 192 | BNGL619 | U | 41 | 2.54 | 1.19 |
| 9 | 196 | UMC2131 | A | 46 | 2.41 | 1.17 |
| 9 | 196 | UMC2131 | C | 25 | 1.80 | 0.91 |
| 10 | 9 | PHI041 | A | 36 | 2.47 | 1.11 |
| 10 | 9 | PHI041 | F | 20 | 1.70 | 1.03 |

TABLE 7A
Markers Significantly Associated with Plant Regeneration
in Hi-II × Gaspe Flint Double Haploid Lines

| | | Marker | Sample | F | P |
|---|---|---|---|---|---|
| Chromosome | Position | Name | Size | Value | Value |
| 1 | 231 | MARKER A | 21 | 3.39 | 0.08 |
| 1 | 287 | UMC1991 | 14 | 4.50 | 0.06 |
| 1 | 330 | UMC1774 | 17 | 3.53 | 0.08 |
| 2 | 14 | UMC2245 | 22 | 3.52 | 0.08 |
| 2 | 29 | UMC1265 | 18 | 4.74 | 0.04 |
| 2 | 45 | UMC1934 | 17 | 9.71 | 0.01 |
| 2 | 256 | PHI427434 | 21 | 3.73 | 0.07 |
| 5 | 161 | UMC2305 | 23 | 3.50 | 0.05 |
| 7 | 10 | UMC1642 | 13 | 5.20 | 0.04 |
| 7 | 68 | UMC1433 | 16 | 7.47 | 0.02 |
| 7 | 184 | UMC1125 | 23 | 4.11 | 0.06 |
| 8 | 113 | UMC1858 | 20 | 3.30 | 0.09 |
| 8 | 172 | MARKER C | 21 | 4.27 | 0.05 |
| 9 | 33 | UMC1170 | 19 | 8.11 | 0.00 |
| 9 | 192 | BNGL619 | 21 | 3.14 | 0.07 |
| 9 | 196 | UMC2131 | 21 | 8.69 | 0.01 |
| 10 | 94 | UMC1246 | 18 | 3.58 | 0.08 |

TABLE 7B
Allele Types and Allele Phenotype Means from Table 7A

| | | Marker | | Sample | Trait | Trait |
|---|---|---|---|---|---|---|
| Chromosome | Position | Name | Allele | Size | Mean | SD |
| 1 | 231 | MARKER A | D | 11 | 1.27 | 0.47 |
| 1 | 231 | MARKER A | E | 10 | 1.00 | 0.00 |
| 1 | 287 | UMC1991 | B | 7 | 1.43 | 0.53 |
| 1 | 287 | UMC1991 | C | 7 | 1.00 | 0.00 |
| 1 | 330 | UMC1774 | A | 8 | 1.00 | 0.00 |
| 1 | 330 | UMC1774 | B | 9 | 1.33 | 0.50 |
| 2 | 14 | UMC2245 | F | 7 | 1.71 | 1.11 |
| 2 | 14 | UMC2245 | G | 15 | 1.13 | 0.35 |
| 2 | 29 | UMC1265 | F | 14 | 1.14 | 0.36 |
| 2 | 29 | UMC1265 | G | 4 | 2.00 | 1.41 |
| 2 | 45 | UMC1934 | B | 6 | 1.50 | 0.55 |
| 2 | 45 | UMC1934 | E | 11 | 1.00 | 0.00 |
| 2 | 256 | PHI427434 | A | 9 | 1.67 | 1.00 |
| 2 | 256 | PHI427434 | C | 12 | 1.08 | 0.29 |
| 5 | 161 | UMC2305 | A | 5 | 1.20 | 0.45 |
| 5 | 161 | UMC2305 | D | 4 | 2.00 | 1.41 |
| 5 | 161 | UMC2305 | G | 14 | 1.07 | 0.27 |
| 7 | 10 | UMC1642 | A | 3 | 1.67 | 0.58 |
| 7 | 10 | UMC1642 | D | 10 | 1.10 | 0.32 |
| 7 | 68 | UMC1433 | A | 3 | 2.33 | 1.53 |
| 7 | 68 | UMC1433 | B | 13 | 1.15 | 0.38 |
| 7 | 184 | UMC1125 | B | 11 | 1.55 | 0.93 |
| 7 | 184 | UMC1125 | D | 12 | 1.00 | 0.00 |
| 8 | 113 | UMC1858 | A | 8 | 1.00 | 0.00 |
| 8 | 113 | UMC1858 | C | 12 | 1.58 | 0.90 |
| 8 | 172 | MARKER C | A | 10 | 1.30 | 0.48 |
| 8 | 172 | MARKER C | B | 11 | 1.00 | 0.00 |
| 9 | 33 | UMC1170 | A | 9 | 1.11 | 0.33 |
| 9 | 33 | UMC1170 | F | 1 | 2.00 | 0.00 |
| 9 | 33 | UMC1170 | G | 9 | 1.00 | 0.00 |
| 9 | 192 | BNGL619 | N | 10 | 1.40 | 0.52 |
| 9 | 192 | BNGL619 | T | 1 | 1.00 | 0.00 |
| 9 | 192 | BNGL619 | U | 10 | 1.00 | 0.00 |
| 9 | 196 | UMC2131 | A | 12 | 1.00 | 0.00 |
| 9 | 196 | UMC2131 | C | 9 | 1.44 | 0.53 |
| 10 | 94 | UMC1246 | A | 10 | 1.10 | 0.32 |
| 10 | 94 | UMC1246 | B | 8 | 1.75 | 1.04 |

### EXAMPLE 5

### Construction and Generation of Doubled Haploid Lines from F2 of PHWWD and PH09B

PHWWD (U.S. patent application Ser. No. 11/431,789) is a doubled haploid line and it is derived from Hi-II and PH09B. PHWWD can produce a Type II callus similar to Hi-II. The callus is very friable, fast growing and highly regenerable. It is also very similar to Hi-II for its transformation efficiency rate. With Agrobacterium, the transformation frequency ranges from 43.5% (with bar as the selection gene) to 53.9% (with GAT as the selection gene). With gun bombardment, the transformation frequency is 35%. The transformation efficiency rates of PHWWD are comparable to the transformation efficiency rates of Hi-II. Therefore, for analysis it is assumed that PHWWD possesses all genetic components from Hi-II that are responsible for T-DNA infection, tissue culture traits and transformation efficiency rates.

PH09B is an elite maize line described in U.S. Pat. No. 5,859,354. PH09B has very low transformation efficiency rates. The transformation frequency of PH09B with Agrobacterium was zero percent and the transformation frequency of the F1 of Hi-II x PH09B is less than 0.3%.

Molecular markers were used to analyse the genetic components of PHWWD. Four hundred and fifty SSR markers that showed to be polymorphic between PH09B and Hi-II were used for this analysis. By using markers it is estimated that the PHWWD genome, contains about 39% of its genome from Hi-II and about 61% of its genome from PH09B. The marker data indicated the origins (either from PH09B or Hi-II) of different proportions of the chromosomal regions on each of the 10 maize chromosomes.

TABLE 8
SSR Profile Data for PHWWD

| | Marker | |
|---|---|---|
| Bin | Name | Base Pairs |
| 1 | umc1041 | 327 |
| 1 | umc1354 | 309.65 |
| 1.01 | phi056 | 255.3 |
| 1.01 | umc1071 | 117 |
| 1.01 | umc1177 | 107.7 |
| 1.01 | umc1269 | 344.475 |
| 1.01 | umc1484 | 211.5 |
| 1.01 | umc2012 | 73.825 |
| 1.01 | umc2224 | 354.695 |
| 1.03 | umc1701 | 117.675 |
| 1.04 | umc1452 | 360.9 |
| 1.04 | umc2112 | 311.5 |
| 1.04 | umc2217 | 163.75 |
| 1.05 | umc1244 | 348.275 |
| 1.05 | umc1297 | 159.85 |
| 1.05 | umc1689 | 149.5 |
| 1.05 | umc1734 | 251 |
| 1.05 | umc2025 | 131.35 |
| 1.05 | umc2232 | 139.1 |
| 1.06 | umc1396 | 169.1 |
| 1.06 | umc1508 | 246.5 |
| 1.06 | umc1668 | 146.25 |
| 1.06 | umc1709 | 350.65 |
| 1.06 | umc1754 | 224.9 |
| 1.06 | umc1924 | 161.35 |
| 1.06 | umc2234 | 150.5 |
| 1.07 | phi002 | 73.53 |
| 1.07 | umc1128 | 226.9 |
| 1.07 | umc1245 | 305.4 |
| 1.07 | umc1833 | 136.3 |
| 1.07 | umc2237 | 162.05 |
| 1.08 | umc1446 | 161.3 |
| 1.08 | umc2385 | 264.35 |
| 1.09 | umc1298 | 362.65 |
| 1.09 | umc1715 | 152.5 |
| 1.09 | umc2047 | 133.25 |
| 1.1 | umc1885 | 145.875 |
| 1.1 | umc2149 | 152.375 |
| 1.11 | umc1553 | 276 |
| 1.11 | umc1737 | 350.5 |
| 1.11 | umc1862 | 143.05 |
| 1.11 | umc2241 | 333.1 |
| 1.11 | umc2242 | 382 |
| 2 | umc1419 | 106.7 |
| 2 | umc2245 | 150.1 |
| 2.02 | umc1518 | 222.5 |
| 2.02 | umc1961 | 309.05 |
| 2.03 | bnlg1621 | 188 |
| 2.04 | phi083 | 125.56 |
| 2.04 | umc1024 | 326.05 |
| 2.04 | umc1026 | 123.95 |
| 2.04 | umc1410 | 214.175 |
| 2.04 | umc1465 | 394.75 |
| 2.04 | umc1541 | 320.525 |
| 2.04 | umc2030 | 168.5 |
| 2.04 | umc2125 | 138.15 |
| 2.04 | umc2247 | 254.6 |
| 2.04 | umc2248 | 154.125 |
| 2.05 | umc1459 | 95.45 |
| 2.06 | umc1658 | 142.1 |
| 2.06 | umc1749 | 206.1 |
| 2.06 | umc1875 | 146 |
| 2.06 | umc2023 | 146.925 |
| 2.06 | umc2192 | 335 |
| 2.06 | umc2254 | 105.95 |
| 2.07 | umc1108 | 205.3 |
| 2.07 | umc1554 | 326.825 |
| 2.07 | umc1637 | 120.6 |
| 2.07 | umc2205 | 174.95 |
| 2.07 | umc2374 | 263 |
| 2.08 | phi090 | 146.005 |
| 2.08 | umc1230 | 310.1 |
| 2.08 | umc1526 | 105 |
| 2.08 | umc1745 | 216 |
| 2.09 | umc1551 | 240.75 |
| 3 | umc2118 | 319.3 |
| 3.01 | umc1394 | 244.3 |
| 3.01 | umc2071 | 150.5 |
| 3.01 | umc2256 | 165.5 |
| 3.01 | umc2376 | 149.5 |
| 3.02 | umc1458 | 335.15 |
| 3.02 | umc1886 | 155.3 |
| 3.04 | umc1030 | 240 |
| 3.04 | umc1347 | 228.35 |
| 3.04 | umc1392 | 148.7 |
| 3.04 | umc1495 | 105.6 |
| 3.04 | umc1908 | 133.6 |
| 3.04 | umc2002 | 125.725 |
| 3.04 | umc2117 | 355.75 |
| 3.04 | umc2263 | 393.4 |
| 3.05 | phi053 | 166.74 |
| 3.05 | phi073 | 187.785 |
| 3.05 | umc1307 | 134.05 |
| 3.05 | umc1400 | 464.6 |
| 3.05 | umc2265 | 203.275 |
| 3.06 | umc1027 | 201.05 |
| 3.06 | umc1311 | 212 |
| 3.06 | umc1644 | 154.95 |
| 3.06 | umc1949 | 112.225 |
| 3.06 | umc1985 | 257.875 |
| 3.06 | umc2270 | 139.85 |
| 3.07 | umc1286 | 234.05 |
| 3.07 | umc1528 | 120.875 |
| 3.07 | umc1690 | 166.5 |
| 3.07 | umc1825 | 160.1 |
| 3.07 | umc2273 | 233.95 |
| 3.08 | umc1273 | 205.825 |
| 3.08 | umc1844 | 142.75 |
| 3.08 | umc2276 | 135.2 |
| 4.01 | phi072 | 139.43 |
| 4.05 | umc1317 | 113.8 |
| 4.05 | umc1390 | 133.5 |
| 4.05 | umc1451 | 109.05 |
| 4.05 | umc1791 | 153.425 |
| 4.05 | umc1851 | 138.5 |
| 4.05 | umc1895 | 147.875 |
| 4.05 | umc1969 | 105.45 |
| 4.05 | umc2061 | 137.35 |
| 4.06 | bnlg2291 | 178.925 |
| 4.06 | bnlg252 | 165.925 |
| 4.06 | umc1702 | 95 |
| 4.06 | umc1869 | 151.5 |
| 4.06 | umc1945 | 113.5 |
| 4.06 | umc2027 | 116.525 |
| 4.07 | umc1620 | 148.35 |
| 4.07 | umc1651 | 95.625 |
| 4.07 | umc1847 | 160.15 |
| 4.08 | bnlg1927 | 198.9 |
| 4.08 | umc1051 | 125.9 |
| 4.08 | umc1132 | 132.5 |
| 4.08 | umc1559 | 141.35 |
| 4.08 | umc1667 | 147 |
| 4.08 | umc1856 | 156.9 |
| 4.08 | umc1871 | 135.5 |
| 4.09 | umc1101 | 137.6 |
| 4.09 | umc1650 | 137 |
| 4.09 | umc1740 | 98.35 |
| 4.09 | umc1834 | 163.425 |
| 4.09 | umc1940 | 128.5 |
| 4.09 | umc1999 | 125.8 |
| 4.09 | umc2046 | 115.8 |
| 4.09 | umc2139 | 138.775 |
| 5 | umc1445 | 225.1 |
| 5 | umc1491 | 248.275 |
| 5 | umc2022 | 153.5 |
| 5 | umc2292 | 137.675 |
| 5.01 | phi024 | 361.6 |
| 5.01 | umc1365 | 115.05 |
| 5.01 | umc1894 | 159.325 |
| 5.02 | umc1587 | 143.6 |
| 5.03 | umc1731 | 364.7 |
| 5.03 | umc1830 | 196.35 |
| 5.03 | umc2297 | 151 |
| 5.03 | umc2400 | 211.6 |
| 5.04 | umc1060 | 231.075 |
| 5.04 | umc1221 | 148.35 |
| 5.04 | umc1332 | 205.75 |
| 5.04 | umc1629 | 114.5 |
| 5.04 | umc1815 | 274.5 |
| 5.04 | umc1990 | 132.75 |
| 5.04 | umc2302 | 348.45 |
| 5.05 | umc1348 | 226 |
| 5.05 | umc1482 | 216.1 |
| 5.05 | umc1800 | 154.15 |
| 5.05 | umc1822 | 103 |
| 5.06 | phi085 | 233.635 |
| 5.06 | umc1941 | 122 |
| 5.06 | umc2198 | 166.25 |
| 5.06 | umc2305 | 164.35 |
| 5.07 | umc2013 | 131.4 |
| 5.08 | umc1225 | 109.75 |
| 5.08 | umc1792 | 120.725 |
| 5.09 | umc1153 | 105.225 |
| 5.09 | umc2209 | 167.8 |
| 6 | umc1002 | 123.3 |
| 6 | umc1018 | 349.7 |
| 6 | umc1883 | 86.175 |
| 6.01 | phi077 | 125 |
| 6.01 | umc1186 | 268.675 |
| 6.01 | umc1195 | 138.175 |
| 6.01 | umc1229 | 215.85 |
| 6.05 | umc1020 | 136.5 |
| 6.05 | umc1114 | 210.875 |
| 6.06 | umc1424 | 293.95 |
| 6.07 | phi070 | 78.235 |
| 6.07 | umc1350 | 123 |
| 6.07 | umc1490 | 258.5 |
| 6.07 | umc1621 | 209.6 |
| 6.07 | umc1653 | 244.475 |
| 6.08 | umc2059 | 147.875 |
| 7 | umc1241 | 121.25 |
| 7 | umc1642 | 153.4 |
| 7.02 | umc1068 | 341 |
| 7.02 | umc1393 | 259.5 |
| 7.02 | umc1401 | 159.35 |
| 7.02 | umc1978 | 115.25 |
| 7.02 | umc2057 | 156.075 |
| 7.03 | umc1841 | 109.15 |
| 7.03 | umc1001 | 145.25 |
| 7.03 | umc1134 | 321.225 |
| 7.03 | umc1275 | 314.1 |
| 7.03 | umc1324 | 212.175 |
| 7.03 | umc1450 | 130.35 |
| 7.03 | umc1456 | 128 |
| 7.03 | umc1567 | 323.2 |
| 7.03 | umc1865 | 151.8 |
| 7.04 | umc1125 | 190.425 |
| 7.04 | umc1342 | 231.45 |
| 7.04 | umc1412 | 156.025 |
| 7.04 | umc1710 | 246.355 |
| 7.04 | umc1799 | 104.55 |
| 7.05 | umc1154 | 261.15 |
| 7.05 | umc1760 | 224.3 |
| 7.06 | phi116 | 165.04 |
| 8.01 | umc1075 | 243.875 |
| 8.01 | umc1483 | 310.75 |
| 8.01 | umc1786 | 353.7 |
| 8.02 | umc1304 | 251.5 |
| 8.02 | umc1790 | 153.5 |
| 8.02 | umc1872 | 148.5 |
| 8.02 | umc1974 | 485.7 |
| 8.02 | umc2004 | 95.675 |
| 8.03 | phi115 | 302.625 |
| 8.03 | phi121 | 98.165 |
| 8.03 | umc1034 | 137 |
| 8.03 | umc1457 | 339.45 |
| 8.03 | umc1470 | 348.9 |
| 8.03 | umc1741 | 160.95 |
| 8.03 | umc1910 | 161.25 |
| 8.05 | umc1562 | 239.7 |
| 8.08 | phi015 | 100.105 |
| 8.09 | umc1638 | 141 |
| 9.01 | umc1588 | 323 |
| 9.01 | umc1596 | 106.45 |
| 9.01 | umc1809 | 230.325 |
| 9.01 | umc2362 | 167.55 |
| 9.02 | umc1636 | 181.7 |
| 9.02 | umc2336 | 258.4 |
| 9.03 | bnlg127 | 222.5 |
| 9.03 | phi022 | 240.55 |
| 9.03 | umc1420 | 316.95 |
| 9.03 | umc1691 | 142 |
| 9.03 | umc1743 | 134 |
| 9.03 | umc2337 | 139.35 |
| 9.03 | umc2370 | 133.4 |
| 9.04 | umc1267 | 342.275 |
| 9.04 | umc1522 | 252.95 |
| 9.04 | umc2394 | 366.35 |
| 9.04 | umc2398 | 126.25 |
| 9.05 | umc1357 | 251 |
| 9.05 | umc1519 | 220.25 |
| 9.05 | umc1657 | 164.35 |
| 9.05 | umc2341 | 130.3 |
| 9.05 | umc2371 | 151.6 |
| 9.06 | umc2346 | 300.5 |
| 9.07 | bnlg1375 | 117.75 |
| 9.07 | umc1104 | 216.925 |
| 9.07 | umc1505 | 142.175 |
| 9.07 | umc2089 | 137.5 |
| 9.07 | umc2131 | 264.475 |
| 10 | umc1293 | 161.275 |
| 10.01 | umc1318 | 216.5 |
| 10.01 | umc2053 | 100.8 |
| 10.02 | umc1152 | 162.5 |
| 10.02 | umc1432 | 119.05 |
| 10.02 | umc1582 | 274.5 |
| 10.02 | umc2034 | 132.55 |
| 10.02 | umc2069 | 374.95 |
| 10.03 | umc1345 | 166.5 |
| 10.03 | umc1785 | 218 |
| 10.03 | umc1938 | 154.5 |
| 10.03 | umc2016 | 125.475 |
| 10.03 | umc2067 | 152 |
| 10.04 | phi062 | 157.805 |
| 10.04 | umc1115 | 329.95 |
| 10.04 | umc1272 | 206.5 |
| 10.04 | umc1280 | 432.225 |
| 10.04 | umc1330 | 340.275 |
| 10.04 | umc1648 | 144 |
| 10.04 | umc1678 | 154.5 |
| 10.04 | umc1930 | 102.6 |
| 10.04 | umc2003 | 96.4 |
| 10.05 | umc1506 | 168.65 |
| 10.06 | umc1045 | 173.5 |
| 10.06 | umc1249 | 242 |
| 10.06 | umc1993 | 108.7 |
| 10.07 | umc1176 | 348.5 |
| 10.07 | umc1344 | 210.755 |
| 10.07 | umc1569 | 234.575 |
| 10.07 | umc1640 | 103.925 |
| 10.07 | umc1645 | 165.8 |
| 10.07 | umc2021 | 135.5 |

Since PHWWD possesses a similar transformability rate as Hi-II in terms of Agrobacterium infection, callus type and quality, plant regeneration capabilities and transformation frequency etc. and PH09B is very difficult to transform and often not transformable, it is assumed for analysis purposes that PHWWD contains all of the genes from Hi-II that are responsible for genetic transformation.

To map the chromosomal loci that contribute to genetic transformation in maize within the 39% of the Hi-II chromosomal regions transferred to PHWWD, a new population of doubled haploid lines was created. First, a cross was made between PHWWD and PH09B. PHWWD was used as the female parent and PH09B was used as the male parent to produce the F1 seeds. Second, the F1 seeds were planted and the silks of the resulted F1 plants were pollinated with pollen from haploid inducer line—RWS-GFP (GFP is a marker gene producing visible green florescent protein) (U.S. patent application Ser. No. 11/298,973). Immature embryos from these F1 ears were isolated and placed on the embryos rescue medium. Under a florescent microscope, some embryos showed green color due to GFP expression and some embryos showed regular embryo color due to lack of GFP expression. Those embryos lacking GFP expression were haploid embryos. These haploid immature embryos were germinated on the medium containing chromosome doubling agent, such as colchicine or pronamide. The germinated plantlets were transplanted to soil in pots and grow in greenhouse. When these plants produced pollen and silks, these plants were self-pollinated to produce seeds. The seeds produced from each doubled haploid plant were homozygous and were considered doubled haploid seeds. The detailed technology was described in U.S. patent application Ser. No. 11/532,921. Through this process, seeds from more than 658 doubled haploid plants were produced. All of the progeny derived from a single doubled haploid plant were designated as a doubled haploid line.

PHWWD contains 61% of PH09B genetic background so the F1 generation of a cross between PHWWD and PH09B should contain about 80% of the PH09B genome. And the average PH09B background in the doubled haploid lines derived from these F1 seeds should also be about 80%.

The genetic components of these doubled haploid lines are equivalent to the F2 generation of PHWWD x PH09B. The 39% of the Hi-II genetic components in PHWWD are randomly distributed in all of these 658 doubled haploid lines. Different proportions of the 39% Hi-II background were contained in each doubled haploid line via genetic recombination. This provided an ideal population to map the genetic loci that are responsible for genetic transformation in maize.

Molecular markers were used to analyse the genetic make-up in each of these 658 doubled haploid lines. The molecular marker data showed that these 658 doubled haploid lines have a normal distribution pattern of the PH09B genetic background. The PH09B background in these doubled haploid lines ranges from 65% to 95%. The data confirmed that these 658 doubled haploid lines generated through haploid technology provided a random distribution of genetic components just as an F2 population derived from an F1 self-pollination would.

These doubled haploid lines were planted in the field. Each line was planted in one row (about 20 plants) and the plants derived from each doubled haploid line were evaluated for a uniform phenotype from seedling stage to maturation. Phenotype characteristics noted included plant shape, plant height, ear height, silk color, tassel shape, another color, maturation date, cob color and kernel color etc. These data were used to confirm that these 658 doubled haploid lines were homozygous.

Through these processes, the population was constructed for mapping the genetic loci related to maize transformability.

### EXAMPLE 6

### Phenotyping of these Doubled Haploid Lines for Genetic Transformability

These 658 doubled haploid lines were evaluated for their Agrobacterium-mediated transformability as well as general tissue culture characterization.

Seeds from each doubled haploid line were planted in the greenhouse and the resulting plants were self-pollinated to produce immature kernels. Immature embryos are isolated from each doubled haploid line to initiate the evaluation process. Usually about 50 immature embryos from each doubled haploid line were used for Agrobacterium-mediated transformation evaluations and 20 immature embryos from each doubled haploid line were used for tissue culture characterization without Agrobacterium infection.

The immature embryos isolated from 9 Hi-II plants and 13 PHWWD plants grown in the greenhouse along with these doubled haploid lines were used as the controls for both Agrobacterium-mediated transformation evaluation and tissue culture characterization without Agrobacterium infection.

The protocol of Agrobacterium-mediated transformation was described in the U.S. Pat. No. 5,981,840 and the publication of Zuo-yu Zhao, Weining Gu, Tishu Cai, Laura Tagliani, Dave Hondred, Diane Bond, Sheryl Schroeder, Marjorie Rudert and Dorothy Pierce; “High throughput genetic transformation mediated by Agrobacterium tumefaciens in maize”; Molecular Breeding, 8 (4): 323-333, 2001.

The T-DNA in the Agrobacterium cell contained two marker genes—the maize ubiquitin (Ubi) promoter driving a GFP gene as the visible marker and the 35S promoter driving a bar gene as the selection marker. The second intron from the potato ST LS1 gene was inserted into the coding region to produce intron-GFP, in order to prevent GFP expression in Agrobacterium cells.

Fifteen traits were fully evaluated. These 15 traits were divided into three major groups. Group-1: Agrobacterium-infected embryos including A) T-DNA delivery, B) Callus initiation frequency, C) Callus type & quality, D) Callus growth rate, E) Callus transformation frequency, F) Regeneration quality, and G) Regeneration frequency. Group-2: non-Agrobacterium-infected embryos including H) Callus initiation frequency, I) Callus type & quality, J) Callus response frequency, K) Callus growth rate, L) Regeneration quality, and M) Regeneration frequency. Group-3: Combining both the Agrobacterium-infected and the non-Agrobacterium infected embryos including N) Agrobacterium hypersensitive response (callus initiation frequency) and O) Agrobacterium hypersensitive response (callus response frequency).

Among these 15 traits, 11 traits (B-D, F-M) are tissue culture related traits and 4 traits (A, E, N and O) are related to interaction of Agrobacterium and plant cells.

These traits were assessed in detail and each assessment was recorded for individual doubled haploid lines.

Agrobacterium-Infected Immature Embryos for Transformation Evaluations:

A. T-DNA Delivery:

Capability of immature embryos receiving T-DNA was based on the transient gene expression of the visible marker gene—GFP in immature embryos following Agrobacterium infection of the immature embryos. At the 3^{rd} day following Agrobacterium infection of the immature embryos, the GFP expression in the immature embryos is scored. All of the embryos from one doubled haploid line were scored together as an average score. Immature embryos from Hi-II and PHWWD were used as positive controls and immature embryos from PH09B were used as the negative control.

Score 1=High T-DNA delivery, score 2=Medium T-DNA delivery, score 3=Low T-DNA delivery, score 4=Very Low T-DNA delivery and score 5=No T-DNA delivery.

Criteria of these Scores:

Medium T-DNA delivery: The positive controls (Hi-II and PHWWD) are defined as Medium T-DNA delivery and any doubled haploid lines showing similar GFP spots on their embryos were scored as Medium for this trait.

High T-DNA delivery: ˜30% or more GFP spots on the immature embryos than Hi-II and/or PHWWD were defined as High T-DNA delivery.

Low T-DNA Delivery: 30-50% less GFP spots on the immature embryos than Hi-II and/or PHWWD were defined as Low T-DNA delivery.

Very Low T-DNA delivery: only a few GFP spots (less than 10 tiny spots on each embryo) on each immature embryo were defined as Very low T-DNA delivery.

No T-DNA delivery: no visible GFP spot on the immature embryos was defined as No T-DNA delivery.

B. Callus Initiation Frequency:

Following Agrobacterium infection and co-cultivation, the embryos were cultured on callus induction medium containing herbicide selection agent. The embryos were sub-cultured every 2 weeks. Callus initiation frequency was calculated at the end of the sixth week. Callus initiation frequency is the number of embryos initiating callus response divided by the total number of embryos culture from each doubled haploid line.

C. Callus Type & Quality:

In maize tissue culture, two major types of callus are clearly defined, Type I and Type II. In general, Type I callus is compact and slow-growing callus and Type II callus is friable and fast-growing callus. Hi-II embryos produce very friable and fast-growing embryogenic Type II callus tissue and PH09B embryos produce a low frequency of Type I callus.

The quality of the callus was scored based on the uniformity of the callus produced from the group of embryos in each doubled haploid line, the maintainability of the callus on medium and embryogenesis capability of the callus. It is scored at ninth week following Agrobacterium infection.

Score 1=High-Quality Type II, score 2=Medium-Quality Type II, score 3=Mixed Type I & II, score 4=Type I, score 5=Low Quality Callus, score 6=No Callus Response.

Criteria of these Scores:

High-Quality Type II: fast-growth, friable and uniform Type II, similar to Hi-II or PHWWD callus.

Medium-Quality Type II: Type II with less than 30% non-embryogenic callus, but it is still good Type II callus for transformation.

Mixed Type I & II: Type I callus is 30%-50% and Type II callus is 50-70%. In general, the callus is still okay for transformation.

Type I: If more than 50% of callus is Type I, it is scored as Type I.

Low Quality Callus: If the callus has a significant amount of non-regenerable tissues (more than 70% of the total callus), such as rooting or watery tissues, it was scored as Low Quality Callus.

No Callus Response: if the embryos can not initiate callus or initiated and stopped shortly, it is scored as No Callus Response.

D. Callus Growth Rate:

Callus growth rate is one of the important factors for genetic transformation through embryogenic tissue culture. During cell division, DNA is replicated and foreign DNA (transgenic genes) can be incorporated into plant genome to produce transgenic cells. Callus Growth Rate was scored at ninth week following Agrobacterium infection. The Callus Growth Rate was based on the average size of the callus from all embryos isolated from each doubled haploid line. The average callus size of the embryos from Hi-II and PHWWD was used as the standard for comparison.

Score 1=Very Fast, score 2=Fast, score 3=Medium, score 4=Slow, and score 5=Very Slow.

Criteria of these Scores:

Very Fast: the average size of the callus tissue was 20% or more larger than Hi-II and PHWWD callus tissues.

Fast: similar to the callus tissues of Hi-II and PHWWD.

Slow: the average size of the callus tissues was 40% to 80% less than Hi-II and PHWWD.

Medium: between Fast and Slow.

Very Slow: the average size of callus tissues was >80% less than Hi-II and PHWWD including the embryos no callus response.

E. Callus Transformation Frequency:

Stable callus transformation was determined based on the expression of the visible marker gene, GFP, in callus tissue at the ninth week following Agrobacterium infection. The score was as the number of embryos producing stable transformed callus (GFP+) divided by the total embryos infected.

F. Regeneration Quality:

Plant regeneration capability is another important factor for plant genetic transformation. Two major steps are involved in embryogenesis in plants. The conversion from callus tissues into somatic embryos is the first step and germination of the somatic embryos into plantlets is the second step for plants regeneration.

Regeneration Quality was used to evaluate these two major steps. After culturing the stably transformed callus tissues on regeneration medium, 1) how easy and quick the callus tissue can convert into somatic embryos and form plantlets and 2) how many of plantlets one-embryo derived callus tissue can produce, were two criteria to measure the quality of regeneration.

Score 1=High Quality, score 2=Medium Quality, score 3=Low Quality and score 4=No regeneration.

Criteria of these Scores:

High Quality: produced plantlets at second week after cultured on regeneration medium and tissue derived from one embryo produces 5 or more plantlets.

Medium Quality: produced plantlets at 2-3 weeks after cultured on regeneration medium and tissue derived from one embryo produces 1-5 plantlets.

Low Quality: produced plantlets later than 3 weeks after cultured on regeneration medium and tissue derived from one embryo produces 1-5 plantlets.

No Regeneration No plantlet produced after cultured on regeneration medium.

G. Regeneration Frequency:

It was defined as the number of stably transformed callus events that regenerated into plantlets divided by the total number of stably transformed callus events cultured on regeneration medium.

Tissue Culture Characterization without Agrobacterium Infection:

H. Callus Initiation Frequency:

Twenty embryos from each doubled haploid line were cultured on callus induction medium without Agrobacterium infection and were sub-cultured every 2 weeks. Callus initiation frequency was calculated at fourth week. Callus initiation frequency was calculated at 4^{th} week of cultures as the number of embryos initiating callus tissues divided by the total number of embryos cultured from each doubled haploid line.

I. Callus Type & Quality:

It is scored twice, first time at the fourth week and second time at the eighth week from initiation of culture. The criteria used for scoring the Agrobacterium-infected embryos were also used for scoring the non-infected embryos.

J. Callus Response Frequency:

Twenty embryos from each doubled haploid line were cultured on callus induction medium without Agrobacterium infection and were sub-cultured every 2 weeks. Callus response frequency was calculated at the eight week in culture as the number of embryos producing callus tissues divided by the total number of embryos cultured from each doubled haploid line.

K. Callus Growth Rate:

Twenty embryos from each doubled haploid line were cultured on callus induction medium without Agrobacterium infection. The callus tissues from each doubled haploid line were weighted twice on a balance at fourth week of cultures and eight week of cultures respectively, and then use the following formula to calculate the callus growth rate.

$$Callus Growth Rate = \frac{\begin{matrix}Callus weight at {8}^{th} week - \\ callus weight at {4}^{th} week\end{matrix}}{Callus weight at {4}^{th} \text{-} week}$$

Score 1=Very Fast, score 2=Fast, score 3=Medium, score 4=Slow, and Score 5=Very Slow.
The callus growth rate of the embryos from Hi-II and PHWWD was used as the control for scoring.
Criteria of these Scores:
Very Fast=a callus growth rate 10% greater than the callus growth rate of Hi-II and PHWWD was scored as 1.
Fast=a callus growth rate equal to callus growth rate of Hi-II and PHWWD or 1-9% more than the callus growth rate of Hi-II and PHWWD was scored as 2.
Medium=a callus growth rate that was up to 40% less than the callus growth rate of Hi-II and PHWWD was scored as 3.
Slow=a callus growth rate that was 41-70% less than the callus growth rate of Hi-II and PHWWD was scored as 4.
Very Slow=a callus growth rate that was >70% less than the callus growth rate of Hi-II and PHWWD was scored as 5.
L. Regeneration Quality:

Same as (F.) above.

M. Regeneration Frequency:

Same as (G.) above.

Another two traits are related to both Agrobacterium-infected and non-infected embryos.

N. Agrobacterium Hypersensitive Response-IN:

Since Agrobacterium is a plant pathogen, maize immature embryos from some genotypes show hypersensitive response to Agrobacterium. After Agrobacterium infection, embryos may be killed by Agrobacterium and these embryos can not produce healthy callus tissues. This is one of the most important factors that inhibit Agrobacterium-mediated plant transformation. Comparing the callus formation frequency of the embryos without Agrobacterium infection to the embryos with Agrobacterium infection provides data to measure the hyper-sensitivity of a particular plant genotype to Agrobacterium infection.

Since two callus formation frequencies were taken; one was recorded at the fourth week after culture initiation of embryos and another was recorded at the eighth week after culture of embryos in the non-Agrobacterium infected embryo cultures; there were two comparisons. The first one was comparing the callus formation frequency at fourth week of culture of the non-Agrobacterium infected embryos to the Agrobacterium infected embryos; this was called Agrobacterium Hypersensitive Response-IN. The second one was comparing the callus formation frequency at the eighth week of culture of the non-Agrobacterium infected embryos to the Agrobacterium infected embryos; this was called Agrobacterium Hypersensitive Response-R.

$$Agrobacterium Hypersensitive Response \text{-} IN = \frac{\begin{matrix}Callus initiation \% at {4}^{th} week of non \text{-} infected embryo - \\ Callus initiation \% of infected embryos\end{matrix}}{Callus initiation \% at {4}^{th} week of non \text{-} infected embryos}$$

If the Agrobacterium Hypersensitive Response-IN=1, it means this doubled haploid line is most hypersensitive to Agrobacterium infection. If Agrobacterium Hypersensitive Response-IN=0, it mean this doubled haploid line is not hypersensitive to Agrobacterium infection. Any number between 1 and 0 shows the different degrees of hypersensitivity.
O. Agrobacterium Hypersensitive Response-R:

$$Agrobacterium Hypersensitive Response \text{-} R = \frac{\begin{matrix}Callus r esponse \% at {8}^{th} week of non \text{-} infected embryo - \\ Callus initiation \% of infected embryos\end{matrix}}{Callus response \% at {8}^{th} week of non \text{-} infected embryos}$$

If the Agrobacterium Hypersensitive Response-IN=1, it means this doubled haploid line is most hypersensitive to Agrobacterium infection. If Agrobacterium Hypersensitive Response-IN=0, it means this doubled haploid line is not hypersensitive to Agrobacterium infection. Any number between 1 and 0 show the different degrees of hypersensitivity.

In the phenotyping work, data for the 15 traits described above were collected from 658 doubled haploid lines.

Agrobacterium-Infected Embryos

Trait-A: T-DNA delivery

| | | % of the |
|---|---|---|
| Score | # DH lines | Total Lines |
| 1 | 21 | 3.2% |
| 2 | 148 | 22.5% |
| 3 | 396 | 60.3% |
| 4 | 77 | 11.7% |
| 5 | 16 | 2.3% |

Trait-B: Callus Initiation %

| Score | # DH lines | % of the Total Lines |
|---|---|---|
| 0% | 589 | 89.5% |
| 1-10% | 46 | 7.0% |
| 11-20% | 9 | 1.4% |
| 21-40% | 10 | 1.5% |
| >40% | 4 | 0.6% |

Trait-C: Callus Type & Quality

| | | % of the |
|---|---|---|
| Score | # DH lines | Total Lines |
| 1 | 11 | 1.7% |
| 2 | 15 | 2.3% |
| 3 | 7 | 1.1% |
| 4 | 14 | 2.1% |
| 5 | 116 | 17.6% |
| 6 | 495 | 75.2% |

Trait-D: Callus Growth Rate

| | | % of the |
|---|---|---|
| Score | # DH lines | Total Lines |
| 1 | 7 | 1.1% |
| 2 | 20 | 3.0% |
| 3 | 23 | 3.5% |
| 4 | 14 | 2.1% |
| 5 | 594 | 90.3% |

Trait-E: Callus Transformation %

| Score | # DH lines | % of the Total Lines |
|---|---|---|
| 0% | 592 | 90.0% |
| 1-10% | 45 | 6.8% |
| 11-15% | 9 | 1.4% |
| 16-20% | 3 | 0.5% |
| 21-30% | 4 | 0.6% |
| 31-40% | 3 | 0.5% |
| >40% | 2 | 0.3% |

Trait-F: Regeneration Quality

| | | % of the |
|---|---|---|
| Score | # DH lines | Total Lines |
| 1 | 20 | 3.0% |
| 2 | 18 | 2.7% |
| 3 | 13 | 2.0% |
| 4 | 15 | 2.3% |
| No data* | 592 | 90.0% |

*Because no callus was produced from the immature embryos in these doubled haploid lines there is no data for plant regeneration in these lines.

Trait-G: Regeneration %

| | | % of the |
|---|---|---|
| Score | # DH lines | Total Lines |
| 0% | 14 | 2.1% |
| 1-40% | 6 | 0.9% |
| 41-79% | 17 | 2.6% |
| 80-94% | 2 | 0.3% |
| 95-100% | 27 | 4.1% |
| No Data | 592 | 90.0% |

Trait-H: Callus Initiation % at 4^{th} Week

| Score | # DH lines | % of the Total Lines |
|---|---|---|
| 0% | 444 | 67.4% |
| 1-20% | 96 | 14.6% |
| 21-40% | 60 | 9.1% |
| 41-70% | 45 | 6.9% |
| >70% | 11 | 1.7% |
| Contaminated | 2 | 0.3% |

Trait-I: Callus Type and Quality

| Score | # DH lines | % of the Total Lines |
|---|---|---|
| 1 | 9 | 1.4% |
| 2 | 38 | 5.8% |
| 3 | 13 | 2.0% |
| 4 | 11 | 1.7% |
| 5 | 316 | 48.1% |
| 6 | 269 | 40.8% |
| Contaminated | 2 | 0.3% |

Trait-J: Callus Response % at 8^{th} Week

| Score | # DH lines | % of the Total Lines |
|---|---|---|
| 0% | 244 | 37.0% |
| 1-20% | 93 | 14.2% |
| 21-40% | 134 | 20.4% |
| 41-60% | 98 | 15.0% |
| 61-80% | 63 | 9.6% |
| >80% | 24 | 3.6% |
| Contaminated | 2 | 0.3% |

Trait-K: Callus Growth Rate

| Score | # DH lines | % of the Total Lines |
|---|---|---|
| 1 | 13 | 2.0% |
| 2 | 37 | 5.6% |
| 3 | 86 | 13.1% |
| 4 | 235 | 35.8% |
| 5 | 285 | 43.2% |
| Contaminated | 2 | 0.3% |

Trait-L: Regeneration Quality

| Score | # DH lines | % of the Total Lines |
|---|---|---|
| 1 | 12 | 1.8% |
| 2 | 51 | 7.8% |
| 3 | 56 | 8.5% |
| 4 | 296 | 45.1% |
| No data | 243 | 36.8% |

Trait-M: Regeneration %

| Score | # DH lines | % of the Total Lines |
|---|---|---|
| 0% | 296 | 45.1% |
| 1-40% | 27 | 4.1% |
| 41-79% | 58 | 8.8% |
| 80-94% | 9 | 1.4% |
| 95-100% | 25 | 3.8% |
| No Data | 243 | 36.8% |

Trait-N: Agrobacterium Hypersensitive Response-IN

| Score | # DH lines | % of the Total Lines |
|---|---|---|
| 0 | 3 | 0.5% |
| 0.01-0.30 | 8 | 1.2% |
| 0.31-0.80 | 19 | 2.9% |
| 0.81-0.99 | 26 | 4.0% |
| 1 | 156 | 23.7% |
| No data | 446 | 67.7% |

Trait-O: Agrobacterium Hypersensitive Response-R

| Score | # DH lines | % of the Total Lines |
|---|---|---|
| 0 | 1 | 0.2% |
| 0.01-0.30 | 4 | 0.6% |
| 0.31-0.80 | 23 | 3.5% |
| 0.81-0.99 | 36 | 5.5% |
| 1 | 348 | 53.0% |
| No data | 246 | 37.3% |

The phenotyping data were combined with genotyping data to develop a genetic map of the chromosomal loci related to genetic transformation in maize.

For the different traits, the data were statistically calculated for the simple correlations coefficients (r) using SAS PROC CORR (SAS Version 9.1, 2003).

Twelve of these 15 traits, T-DNA delivery (T_DNA_delivery_T), Callus Transformation Frequency (Callus_TX-Pcnt_T), Callus Initiation Frequency of Infected Embryos (Callus_initation_Pcnt_T), Callus Type and Quality of Infected Embryos (Callus_Type_quality_T), Regeneration Quality of Infected Embryos (Reg_Quality_T), Regeneration Frequency of Infected Embryos (Reg_Pcnt_T), Callus Initiation Frequency of non-infected Embryos (Callus_Initiation_Pcnt_C), Callus Type and Quality of non-infected Embryos (Callus_Type_quality_C), Callus Growth Rate of non-infected Embryos (Callus_Growth_Rate_C), Callus Response Frequency of non-Infected Embryos (Callus_response_pcnt_C), Regeneration Quality of non-infected Embryos (Reg_Quality_C), Regeneration Frequency of non-infected Embryos (Reg_Pcnt_C) and another three comparisons, difference of Callus Initiation Frequency of non-infected and Infected Embryos (Callus Initiation_Pcnt_Diff), difference of Callus Type and Quality of non-infected and Infected Embryos (Callus_Type_quality_Diff), and difference of Regeneration Frequency of non-Infected and Infected Embryos (Reg_Pcnt_Diff) were statistically calculated. These correlations are listed in Table 9 below.

TABLE 9
Simple Correlation of Traits

| | Data from Agrobacterium Infected Embryos | | | | |
|---|---|---|---|---|---|
| Variable | T_DNA_delivery_T | TX_Pcnt | Callus_initiation_Pcnt_T | Callus_Type_quality_T | Reg_Quality_T |
| T_DNA_delivery_T | 1 | −0.07 | −0.04 | 0.06 | 0.01 |
| Callus_TX_Pcnt_T | −0.07 | 1 | 0.89 | −0.56 | 0.07 |
| Callus_initiation_Pcnt_T | −0.04 | 0.89 | 1 | −0.46 | 0.08 |
| Callus_Type_quality_T | 0.06 | −0.56 | −0.46 | 1 | 0.72 |
| Reg_Quality_T | 0.01 | 0.07 | 0.08 | 0.72 | 1 |
| Reg_Pcnt_T | 0.12 | −0.13 | −0.11 | −0.58 | −0.81 |
| Callus_initiation_Pcnt_C | −0.03 | 0.45 | 0.46 | −0.40 | −0.15 |
| Callus_Type_quality_C | 0.06 | −0.36 | −0.34 | 0.41 | 0.18 |
| Callus_Growth_Rate_C | 0.08 | −0.37 | −0.34 | 0.42 | 0.23 |
| Callus_response_pcnt_C | −0.07 | 0.30 | 0.32 | −0.29 | −0.04 |
| Reg_Quality_C | 0.00 | −0.34 | −0.33 | 0.41 | 0.19 |
| Reg_Pcnt_C | 0.04 | 0.34 | 0.34 | −0.35 | −0.10 |
| Callus_Initiation_Pcnt_Diff | 0.01 | −0.16 | −0.12 | 0.27 | 0.25 |
| Callus_Type_quality_Diff | −0.02 | −0.11 | −0.06 | 0.44 | 0.46 |
| Reg_Pcnt_Diff | −0.07 | −0.37 | −0.36 | −0.24 | −0.56 |

| | | Data from Control Embryos | | |
|---|---|---|---|---|
| Variable | Reg_Pcnt_T | Callus_initiation_Pcnt_C | Callus_Type_quality_C | Callus_Growth_Rate_C |
| T_DNA_delivery_T | 0.12 | −0.03 | 0.06 | 0.08 |
| Callus_TX_Pcnt_T | −0.13 | 0.45 | −0.36 | −0.37 |
| Callus_initiation_Pcnt_T | −0.11 | 0.46 | −0.34 | −0.34 |
| Callus_Type_quality_T | −0.58 | −0.40 | 0.41 | 0.42 |
| Reg_Quality_T | −0.81 | −0.15 | 0.18 | 0.23 |
| Reg_Pcnt_T | 1 | 0.06 | −0.05 | −0.06 |
| Callus_initiation_Pcnt_C | 0.06 | 1 | −0.56 | −0.63 |
| Callus_Type_quality_C | −0.05 | −0.56 | 1 | 0.76 |
| Callus_Growth_Rate_C | −0.06 | −0.63 | 0.76 | 1 |
| Callus_response_pcnt_C | −0.06 | 0.54 | −0.49 | −0.58 |
| Reg_Quality_C | −0.09 | −0.53 | 0.71 | 0.68 |
| Reg_Pcnt_C | 0.04 | 0.47 | −0.68 | −0.59 |
| Callus_Initiation_Pcnt_Diff | −0.15 | −0.94 | 0.50 | 0.57 |
| Callus_Type_quality_Diff | −0.46 | 0.21 | −0.64 | −0.39 |
| Reg_Pcnt_Diff | 0.73 | −0.33 | 0.51 | 0.39 |

| | Data from Control Embryos | | |
|---|---|---|---|
| Variable | Callus_response_pcnt_C | Reg_Quality_C | Reg_Pcnt_C |
| T_DNA_delivery_T | −0.07 | 0.00 | 0.04 |
| Callus_TX_Pcnt_T | 0.30 | −0.34 | 0.34 |
| Callus_initiation_Pcnt_T | 0.32 | −0.33 | 0.34 |
| Callus_Type_quality_T | −0.29 | 0.41 | −0.35 |
| Reg_Quality_T | −0.04 | 0.19 | −0.10 |
| Reg_Pcnt_T | −0.06 | −0.09 | 0.04 |
| Callus_initiation_Pcnt_C | 0.54 | −0.53 | 0.47 |
| Callus_Type_quality_C | −0.49 | 0.71 | −0.68 |
| Callus_Growth_Rate_C | −0.58 | 0.68 | −0.59 |
| Callus_response_pcnt_C | 1 | −0.07 | −0.03 |
| Reg_Quality_C | −0.07 | 1 | −0.86 |
| Reg_Pcnt_C | −0.03 | −0.86 | 1 |
| Callus_Initiation_Pcnt_Diff | −0.48 | 0.46 | −0.38 |
| Callus_Type_quality_Diff | 0.24 | −0.26 | 0.29 |
| Reg_Pcnt_Diff | −0.02 | 0.52 | −0.65 |

| | Data from Control-Infected | | |
|---|---|---|---|
| Variable | Callus_Initiation_Pcnt_Diff | Callus_Type_quality_Diff | Reg_Pcnt_Diff |
| T_DNA_delivery_T | 0.01 | −0.02 | −0.07 |
| Callus_TX_Pcnt_T | −0.16 | −0.11 | −0.37 |
| Callus_initiation_Pcnt_T | −0.12 | −0.06 | −0.36 |
| Callus_Type_quality_T | 0.27 | 0.44 | −0.24 |
| Reg_Quality_T | 0.25 | 0.46 | −0.56 |
| Reg_Pcnt_T | −0.15 | −0.46 | 0.73 |
| Callus_initiation_Pcnt_C | −0.94 | 0.21 | −0.33 |
| Callus_Type_quality_C | 0.50 | −0.64 | 0.51 |
| Callus_Growth_Rate_C | 0.57 | −0.39 | 0.39 |
| Callus_response_pcnt_C | −0.48 | 0.24 | −0.02 |
| Reg_Quality_C | 0.46 | −0.26 | 0.52 |
| Reg_Pcnt_C | −0.38 | 0.29 | −0.65 |
| Callus_Initiation_Pcnt_Diff | 1 | −0.26 | 0.11 |
| Callus_Type_quality_Diff | −0.26 | 1 | −0.67 |
| Reg_Pcnt_Diff | 0.11 | −0.67 | 1 |

The analysis results in Table 9 showed the trait of T-DNA delivery is not correlated to other tissue culture related traits. Callus Transformation Frequency is highly related to Callus Initiation frequency, Callus Type and Quality and Callus Growth Rate etc. All of other tissue culture related traits are correlated at certain degrees.

### EXAMPLE 7

### Genotyping of these Doubled Haploid Lines with Molecular Markers

Since PHWWD has 31% of chromosomal regions from Hi-II and 61% from PH09B and PHWWD has the same or similar capability as Hi-II for genetic transformation; it is assumed that the genetic components that are responsible for transformation are located within these 31% of the Hi-II chromosomal regions in PHWWD. All of the polymorphic regions between PHWWD and PH09B are also located within these 31% of Hi-II regions. The marker analysis of these 658 doubled haploid lines was focused on these 31% of the Hi-II chromosomal regions.

Simple Sequence Repeats (SSR) markers described earlier were used for genotyping of these 658 doubled haploid lines.

The parents of the population—PH09B and PHWWD—were screened to identify the polymorphic markers. Polymorphic markers between these parents were further used for SSR analysis in the population. The polymorphic markers for genome coverage and quality of the markers were taken into consideration. Leaf disks from each seedlings of 4-6 week were collected in 96-well plates. DNA was extracted using a robotic system. SSR genotyping was performed.

### EXAMPLE 8

### Quantitative Trait Locus (QTL) Analysis to Map the Transformability Loci

Using a Pioneer proprietary genetic map (PHD map) and the phenotypic data described above, single marker and composite interval mapping (CIM) was implemented in Windows QTL Cartographer version 2.5 (Wang S., C. J. Basten, and Z.-B. Zeng, 2007; Windows QTL Cartographer 2.5, Department of Statistics, North Carolina State University, Raleigh, N.C. (The world wide web at //statgen.ncsu.edu/qtlcart/WQTLCart.htm) to detect QTLs affecting each trait. The threshold LOD (Logarithmic odds) score at significance level of 0.05 was estimated empirically with 300 permutations (Churchill, G. A., and R. W. Doerge. 1994. Empirical threshold values for quantitative trait mapping. Genetics 138:963-971). Default settings in Windows QTL Cartographer were used for the QTL analysis. The marker data was converted into the IBM2+2005 Neighbors map positions which is publicly available.

Through the QTL mapping of these doubled haploid lines, these 15 traits, A-O were mapped in several chromosomal regions. These traits are listed below.

A. T-DNA Delivery

B. Callus Initiation %—infected

C. Callus T&Q—infected

D. Callus Growth Rate—infected

E. Transformation %

F. Regeneration Q—infected

G. Regeneration %—infected

H. Callus Initiation %—no Agro

I. Callus T&Q—no Agro

J. Callus Response %—no Agro

K. Callus Growth Rate—no Agro

L. Regeneration Q—no Agro

M. Regeneration %—no Agro

N. Agro Hypersensitive-IN

O. Agro Hypersensitive-R

Through QTL mapping, the loci that genetically control these 13 traits are mapped on Regions of chromosome 1, 3, 4, and 5. These regions can be summarized in the following Table 10.

TABLE 10
Transformability traits mapped onto IBM2+ 2005 Neighbors by QTL
mapping

| | Flanking Markers | | | |
|---|---|---|---|---|
| | (name map | | | |
| | position and bin | | | |
| | number) | | | Max |
| | | Right | | LOD |
| Chromosome | Left flanking | flanking | Trait | score |
| 1 | Umc2225 | Umc1711 | D. Callus growth rate-infected | 3.34 |
| | 124.7 | 176.69 | | |
| | 1.02 | 1.02 | | |
| 3 | Umc2258 | Umc1908 | K. Callus growth rate-no Agro | 3.47 |
| | 127.8 | 213.6 | | |
| | 3.03 | 3.04 | | |
| 3 | Umc1908 | Umc2265 | A. T-DNA delivery | 2.69 |
| | 213.6 | 354 | | |
| | 3.04 | 3.05 | | |
| 3 | Umc1167 | Umc2076 | H. Callus Initiation %-no Agro | 7.55 |
| | 319.2 | 461.15 | I. Callus T&Q-no Agro | 7.01 |
| | 3.04 | 3.06 | J. Callus Response %-no Agro | 9.36 |
| | | | B. Callus Initiation %-infected | 3.71 |
| | | | C. Callus T&Q-infected | 9.38 |
| | | | E. Transformation % | 4.88 |
| 3 | Umc1400 | Umc1949 | K. Callus Growth Rate-no Agro | 5.85 |
| | 384.92 | 523.52 | D. Callus Growth Rate-infected | 7.86 |
| | 3.05 | 3.06 | | |
| 4 | Bnlg1189 | Umc1043 | H. Callus Initiation %-no Agro | 8.7 |
| | 428.00 | 455.91 | I. T&Q-no Agro | 6.41 |
| | 4.07 | 4.08 | K. Callus Growth Rate-no Agro | 6.15 |
| | | | M. Regeneration %-no Agro | 3.56 |
| | | | D. Callus Growth Rate-infected | 3.77 |
| 5 | Umc1587 | Bnlg653 | A. T-DNA delivery | 8.24 |
| | 156.9 | 307.01 | | |
| | 5.02 | 5.04 | | |
| 5 | Bnlg653 | PHI333597 | C. Callus T&Q-infected | 4.9 |
| | 307.01 | 394.4 | | |
| | 5.04 | 5.05 | | |
| 5 | Umc1941 | Umc108 | D. Callus Growth Rate-infected | 2.87 |
| | 492.7 | 536.6 | | |
| | 5.06 | 5.07 | | |

### EXAMPLE 9

### Association Mapping of the Transformability Loci to Validate the QTL Mapping Results

To validate the results of QTL mapping, five traits were chosen for linkage-disequilibrium based association mapping.

For linkage-disequilibrium based association mapping, a conditional likelihood-based mapping tool GPA (General Pedigree Association) is used (Guoping Shu, Beiyan Zeng, and Oscar Smith, 2003; Detection Power of Random, Case-Control, and Case-Parent Control Designs for Association Tests and Genetic Mapping of Complex Traits: Proceedings of 15th Annual KSU Conference on Applied Statistics in Agriculture. 15: 191-204).

These five traits used for association mapping are:

T-DNA Delivery

Transformation %

Callus Initiation %—no Agro

Callus T&Q—no Agro

Callus Response %—no Agro

Table 11A-11E below lists chromosomal regions and significant SSR markers identified through association mapping.

Table 11A-11E. Chromosomal regions, significant SSR markers and bin locations mapped by association mapping.

TABLE 11A

| Trait | Chromosome | SSR marker | Bin |
|---|---|---|---|
| A. T-DNA delivery- | 3 | UMC1814 | 3.02 |
| infected | 3 | BNLG1647 | 3.02 |
| | 3 | UMC2258 | 3.03 |
| | 3 | UMC1025 | 3.04 |
| | 3 | UMC1495 | 3.04 |
| | 3 | UMC2260 | 3.04 |
| | 3 | UMC1908 | 3.04 |
| | 3 | MARKER K | |
| | 3 | MARKER 0 | |
| | 3 | UMC2264 | 3.04 |
| | 3 | PHI053 | 3.05 |
| | 3 | UMC1907 | 3.05 |
| | 3 | UMC1167 | 3.04 |
| | 5 | UMC1587 | 5.02 |
| | 5 | UMC1853 | 5.05 |
| | 7 | UMC1125 | 7.04 |

TABLE 11B

| Trait | Chromosome | SSR marker | Bin |
|---|---|---|---|
| E. Transformation % | 3 | UMC1025 | 3.04 |
| | 3 | MARKER N | |
| | 3 | UMC2260 | 3.04 |
| | 3 | MARKER K | |
| | 3 | MARKER O | |
| | 3 | UMC2264 | 3.04 |
| | 3 | PHI053 | 3.05 |
| | 3 | UMC1907 | 3.05 |
| | 3 | UMC1167 | 3.04 |
| | 3 | UMC2265 | 3.05 |
| | 3 | UMC1400 | 3.05 |
| | 3 | MARKER M | |
| | 3 | UMC1985 | 3.06 |
| | 3 | BNLG1160 | 3.06 |
| | 4 | UMC1808 | 4.08 |
| | 5 | UMC1830 | 5.03 |
| | 5 | PHI333597 | 5.05 |
| | 6 | UMC1424 | 6.06 |
| | 7 | UMC1412 | 7.04 |
| | 7 | UMC1125 | 7.04 |

TABLE 11C.

| Trait | Chromosome | SSR marker | Bin |
|---|---|---|---|
| H. Callus initiation %- | 3 | UMC2260 | 3.04 |
| no Agro | 3 | UMC2265 | 3.05 |
| | 3 | UMC1400 | 3.05 |
| | 3 | MARKER M | |
| | 3 | UMC1985 | 3.06 |
| | 3 | BNLG1160 | 3.06 |
| | 3 | UMC1949 | 3.06 |
| | 4 | UMC1667 | 4.08 |
| | 4 | UMC1043 | 4.08 |
| | 4 | PHI314704 | 4.09 |
| | 6 | UMC1114 | 6.05 |
| | 6 | BNLG1174 | 6.05 |
| | 6 | PMG1 | 6.05 |
| | 6 | PHI445613 | 6.05 |
| | 6 | UMC1424 | 6.06 |
| | 8 | UMC1075 | 8.01 |

TABLE 11D.

| Trait | Chromosome | SSR marker | Bin |
|---|---|---|---|
| I. Callus Type & | 3 | BNLG1647 | 3.02 |
| Quality-no Agro | 3 | UMC2258 | 3.03 |
| | 3 | MARKER R | |
| | 3 | UMC1495 | 3.04 |
| | 3 | MARKER N | |
| | 3 | UMC2260 | 3.04 |
| | 3 | UMC1908 | 3.04 |
| | 3 | MARKER O | |
| | 3 | UMC2264 | 3.04 |
| | 3 | PHI053 | 3.05 |
| | 3 | UMC1167 | 3.04 |
| | 3 | UMC2265 | 3.05 |
| | 3 | UMC1400 | 3.05 |
| | 3 | MARKER M | |
| | 3 | UMC1985 | 3.06 |
| | 3 | BNLG1160 | 3.06 |
| | 3 | UMC1949 | 3.06 |
| | 4 | BNLG1189 | 4.07 |
| | 4 | UMC1808 | 4.08 |
| | 4 | UMC1043 | 4.08 |
| | 4 | MARKER L | |
| | 4 | UMC1086 | 4.08 |
| | 4 | MARKER 0 | |
| | 6 | UMC1424 | 6.06 |

TABLE 11E.

| Trait | Chromosome | SSR marker | Bin |
|---|---|---|---|
| J. Callus Response %- | 3 | UMC2265 | 3.05 |
| no Agro | 3 | UMC1400 | 3.05 |
| | 3 | UMC1985 | 3.06 |
| | 3 | BNLG1160 | 3.06 |
| | 3 | UMC1949 | 3.06 |
| | 6 | UMC1114 | 6.05 |
| | 6 | BNLG1174 | 6.05 |
| | 6 | PMG1 | 6.05 |
| | 6 | PH1445613 | 6.05 |
| | 6 | UMC1424 | 6.06 |
| | 8 | UMC1075 | 8.01 |

TABLE 12
As the result of QTL mapping it was shown that these 5 traits
shared some common markers and are mapped in some
overlapping or the same chromosomal regions. Among these
significant SSR markers the following 44 markers are unique
markers for these 5 traits.

| SSR Marker | Bin |
|---|---|
| Marker K | |
| Marker L | |
| PHI314704 | 4.09 |
| PHI333597 | 5.05 |
| Marker M | |
| Marker N | |
| PHI445613 | 6.05 |
| Marker O | |
| Marker Q | |
| Marker R | |
| BNLG1160 | 3.06 |
| BNLG1174 | 6.05 |
| BNLG1189 | 4.07 |
| BNLG1647 | 3.02 |
| PHI053, UMC102 | 3.05 |
| PMG1, INRA, PGAM1, PGAM2 | 6.05 |
| UMC1025 | 3.04 |
| UMC1043 | 4.08 |
| UMC1075 | 8.01 |
| UMC1086 | 4.08 |
| UMC1114 | 6.05 |
| UMC1125 | 7.04 |
| UMC1167 | 3.04 |
| UMC1400 | 3.05 |
| UMC1412 | 7.04 |
| UMC1424 | 6.06 |
| UMC1495 | 3.04 |
| UMC1587 | 5.02 |
| UMC1667 | 4.08 |
| UMC1808 | 4.08 |
| UMC1814 | 3.02 |
| UMC1830 | 5.03 |
| UMC1853 | 5.05 |
| UMC1907 | 3.05 |
| UMC1908 | 3.04 |
| UMC1949 | 3.07 |
| UMC1985 | 3.06 |
| UMC2258 | 3.03 |
| UMC2260 | 3.04 |
| UMC2264 | 3.04 |
| UMC2265 | 3.05 |

Comparing the chromosomal regions mapped by association mapping to the chromosomal regions mapped by QTL mapping for these five traits, most of the traits are mapped in the same or similar chromosomal regions.

### EXAMPLE 10

Epistasis is the interaction between genes whereby one gene interferes or enhance the expression of another gene (Bateson 1907). Many classical quantitative genetic studies have established the importance of epistasis (eg Falconer 1981). Now, with markers, we can begin to examine epistasis in more detail. Epistasis has been found to be important in grain yield components of maize (Ma et al, 2007). Where epistasis, or interactions, occur between QTL, it is extremely important to consider the types of effects when selecting for the trait with markers. A QTL that has a small, or no, main effect can be extremely important in influencing the expression of a QTL of major effect (Wade 1992). If such interactions are not considered, selecting for only the QTL of large effect may not produce the expected phenotypic gain.

Bateson W (1907) The progress of genetics since the rediscovery of Mendel's paper. Progr Rei Bot 1:368.

Falconer D S (1981) Introduction to quantitative genetics, 2^{nd} edition. Longman Press, New York.

Ma X Q, Tang J H, Teng W T, Yan J B, Meng Y J, Li J S. (2007) Epistatic interaction is an important genetic basis of grain yield and its components in maize. Molecular Breeding 20:41-51.

Wade M J (1992) Sewall Wright: gene interaction and the shifting balance theory. Oxf. Surv. Evol. Biol. 8:35-62.

Pair-wise and three-way interactions between markers significantly associated with major QTL were tested using Generalized Linear modeling (Proc GLM) in SAS (SAS Institute) with markers as main and interacting effects. The phenotypic effects of interactions were examined by comparing the trait means for combinations of alleles at each marker locus.

A_Res=Agro Hypersensitive-R

C_GR=Callus Growth Rate—no Agro

C_I=Callus Initiation %—no Agro

C_RG=Regeneration %—no Agro

C_RGQ=Regeneration Q—no Agro

C_Res=Callus Response %—no Agro

C_TQ=Callus T&Q—no Agro

I_GR=Callus Growth Rate—infected

I_I=Callus Initiation %—infected

I_TQ=Callus T&Q—infected

T_DNA=T-DNA Delivery

Trans=Transformation %

TABLE 13
P values for main effects and interactions for UMC1400 (Chr 3) and
BNLG1189 (Chr 4).

| | UMC1400 | BNLG1189 | |
|---|---|---|---|
| | (Chr 3) | (Chr 4) | UMC1400 × BNLG1189 |
| A_Res | 0.0016** | 0.12 | 0.35 |
| C_GR | 0.00004*** | 0.00000*** | 0.07 |
| C_I | 0.00027*** | 0.00000*** | 0.02* |
| C_RG | 0.00752** | 0.00003*** | 0.08 |
| C_RGQ | 0.02* | 0.00033*** | 0.04* |
| C_Res | 0.00009*** | 0.018* | 0.88 |
| C_TQ | 0.00009*** | 0.00000*** | 0.08 |
| I_GR | 0.00038*** | 0.00004*** | 0.014* |
| I_I | 0.00051*** | 0.08 | 0.12 |
| I_TQ | 0.00000*** | 0.02* | 0.0008*** |
| T_DNA | 0.23 | 0.73 | 0.33 |
| Trans | 0.00021*** | 0.06 | 0.04* |

TABLE 14
P values for main effects and interactions for UMC1400 (Chr 3) and
UMC1332 (Chr 5).

| | UMC1400 | UMC1332 | |
|---|---|---|---|
| | (Chr 3) | (Chr 5) | UMC1400 × UMC1332 |
| A_Res | 0.15 | 0.00047*** | 0.33 |
| C_GR | 0.05* | 0.00000*** | 0.84 |
| C_I | 0.65 | 0.00004*** | 0.31 |
| C_RG | 0.86 | 0.001** | 0.2 |
| C_RGQ | 0.61 | 0.003** | 0.16 |
| C_Res | 0.18 | 0.00002*** | 0.94 |
| C_TQ | 0.10 | 0.00001*** | 0.15 |
| I_GR | 0.004** | 0.00002*** | 0.03* |
| I_I | 0.11 | 0.00007*** | 0.14 |
| I_TQ | 0.004** | 0.00000*** | 0.01** |
| T_DNA | 0.00005*** | 0.18 | 0.23 |
| Trans | 0.017* | 0.00004*** | 0.02* |

Table 15A-C. Means for selected traits where significant interactions were detected for BNLG1189 (Chr 4)*UMC1400 (Chr 3) (grouped by number of available datapoints for each trait). The “A” allele is from PH09B. The “B” allele is from PHWWD.

TABLE 15A

| Level of | Level of | | C_I | C_I |
|---|---|---|---|---|
| BNLG1189 | UMC1400 | N | Mean | Std Dev |
| A | A | 97 | 2.8350515 | 10.4808162 |
| A | B | 128 | 6.3203125 | 17.6063399 |
| B | A | 126 | 8.6904762 | 17.5651766 |
| B | B | 106 | 19.6037736 | 23.6818915 |

TABLE 15B

| Level of | Level of | | C_RG | C_RG | C_RGQ | C_RGQ |
|---|---|---|---|---|---|---|
| BNLG1189 | UMC1400 | N | Mean | Std Dev | Mean | Std Dev |
| A | A | 5 | 0.0411 | 0.1503 | 3.8000 | 0.6941 |
| A | B | 84 | 0.0771 | 0.2328 | 3.7738 | 0.6649 |
| B | A | 77 | 0.1089 | 0.2585 | 3.7012 | 0.6701 |
| B | B | 89 | 0.2577 | 0.3493 | 3.3033 | 0.9096 |

TABLE 15C

| | | | | I_TQ | | I_GR | | Trans |
|---|---|---|---|---|---|---|---|---|
| Level of | Level of | | I_TQ | Std | I_GR | Std | Trans | Std |
| BNLG1189 | UMC1400 | N | Mean | Dev | Mean | Dev | Mean | Dev |
| A | A | 97 | 5.7835 | 0.6162 | 5.1340 | 0.4239 | 0.1443 | 1.4214 |
| A | B | 128 | 5.6171 | 0.9059 | 5.0234 | 0.7982 | 0.8671 | 5.0748 |
| B | A | 126 | 5.8809 | 0.4119 | 5.0158 | 0.3996 | 0.0555 | 0.3645 |
| B | B | 107 | 5.1495 | 1.3722 | 4.5420 | 1.2383 | 2.3925 | 6.5224 |

TABLE 16
Means for selected traits where significant interactions were detected for
UMC1332 (Chr 5) * UMC1400 (Chr 3) (grouped by number of available
datapoints for each trait).

| | | | | I_TQ | | I_GR | | Trans |
|---|---|---|---|---|---|---|---|---|
| Level of | Level of | | I_TQ | Std | I_GR | Std | Trans | Std |
| UMC1332 | UMC1400 | N | Mean | Dev | Mean | Dev | Mean | Dev |
| A | A | 137 | 5.8540 | 0.5221 | 5.0875 | 0.3732 | 0.1021 | 1.1961 |
| A | B | 143 | 5.5384 | 0.9697 | 4.8951 | 0.9323 | 0.9930 | 4.0324 |
| B | A | 101 | 5.8316 | 0.4705 | 5.0396 | 0.4454 | 0.0693 | 0.4063 |
| B | B | 111 | 5.0900 | 1.4987 | 4.5225 | 1.3062 | 2.9099 | 8.4590 |

Although the P values for interactions were generally small, this is because the model also included the markers as main effects, so limiting false positive detection of interactions. It is evident that these interactions have a significant biological effect when the mean trait values are examined. For example, for the trait C_I, in the presence of the A allele at BNLG1189 on chromosome 4, changing the A allele to a B allele for UMC1400 on chromosome 3 resulted in an increase in the trait of 3.49. Alternately, changing the A allele to a B allele for BNLG1189 in the presence of the A allele for UMC1400 resulted in an increase in the trait of 5.86. Changing both alleles at both markers from A to B resulted in an increase in C_I of 16.76, i.e., twice the average phenotypic effect of changing alleles at the individual QTL. This is an over-additive interaction, where the sum of both QTLs is more than each alone. While the QTL on chromosome 3 has a large effect, this large effect can only be achieved in combination with the QTL on chromosome 4, i.e., selecting both QTL will result in greater progress.

Such trends in the means were also apparent for the other traits (negative effects of the two QTL were found where a ‘low’ value was beneficial eg for I_TQ where 1 is a good quality score). Even where the P value was not significant, as for C_RG (P=0.08), the means followed a similar trend of a greater phenotypic effect being achieved with both QTL, suggesting that a larger population size with greater power would detect these interactions.

Interactions between the QTL on chromosome 3 and the QTLs on chromosomes 4 and 5 were apparent, even when main effect QTL were not detected. For example, for the % Transformation trait, a QTL of large effect was detected on chromosome 3, but not on chromosome 4 (with interval mapping, although a close to significant QTL was detected with generalized linear modeling at P=0.06). Interaction analyses and examination of means demonstrated that the QTL region on chromosome 4 was important to enhance the effects of the chromosome 3 QTL for % transformation.

What is claimed is:

1. A method of obtaining a maize plant with increased transformability comprising: a) crossing a first maize plant and a second maize plant wherein said first plant has higher transformability than said second plant; b) taking DNA from cells obtained from said cross or from cells of later filial generations of said cross and hybridizing with one or more markers located in a group consisting of bin 1.01, 1.02, 2.01, 2.02, 2.03, 2.04, 3.01, 3.02, 3.04, 4.08, 4.09, 5.03, 5.05, 5.07, 5.08, 6.01, 6.05, 6.06, 6.07, 6.08, 6.09, 7.04, 7.05, 8.03, 8.04, 8.05, 8.06, 8.07, 10.01, 10.02, and 10.03 and; c) selecting a plant wherein said DNA hybridizes with one or more of the markers to obtain a plant with increased transformability when compared to the transformability rate of the second plant.

2. The method of claim 1 wherein the first maize parent is Hi-II.

3. The method of claim 1 wherein the first maize parent is A188.

4. The method of claim 1 wherein the first maize parent is H99.

5. A method of obtaining a maize plant with increased transformability comprising: a) crossing a first maize plant and a second maize plant wherein said first plant has higher transformability than said second plant; b) taking DNA from cells obtained from said cross or from cells of later filial generations of said cross and hybridizing with one or more markers located in a group consisting of between and including umc2225 and umc1711, between and including umc2258 and umc1908, between and including bnlg1189 and umc1043, between and including blng1189 and umc1043, between and including umc1587 and PH1333597, and between and including umc1941 and umc108 and; c) selecting a plant wherein said DNA hybridizes with one or more of the markers to obtain a plant with increased transformability when compared the transformability rate of the second plant.

6. The method of claim 5 wherein the first maize parent is Hi-II.

7. The method of claim 5 wherein the first maize parent is A188.

8. The method of claim 5 wherein the first maize parent is H99.

9. A method of obtaining a maize plant with increased efficiency for T-DNA delivery comprising: a) crossing a first maize plant and a second maize plant wherein said first plant has higher efficiency for T-DNA delivery than said second plant; b) taking DNA from cells obtained from said cross or from cells of later filial generations of said cross and hybridizing with one or more markers located in a group consisting of bin 5.02, 5.03, and 5.04 and; c) selecting a plant wherein said DNA hybridizes with one or more of the markers to obtain a plant with higher efficiency for T-DNA delivery when compared to the efficiency for T-DNA delivery of the second plant.

10. The method of claim 9 wherein the first maize parent is Hi-II.

11. The method of claim 9 wherein the first maize parent is A188.

12. The method of claim 9 wherein the first maize parent is H99.

13. The method of claim 9, further comprising taking DNA from cells obtained from said cross or from cells of later filial generations of said cross and hybridizing with one or more markers located in bin 3.04 or 3.05.

14. A method of obtaining a maize plant with increased callus initiation and quality comprising: a) crossing a first maize plant and a second maize plant wherein said first plant has increased callus initiation and quality than said second plant; b) taking DNA from cells obtained from said cross or from cells of later filial generations of said cross and hybridizing with one or more markers located in a group consisting of bin 4.07, 4.08, and 4.09 and; c) selecting a plant wherein said DNA hybridizes with one or more of the markers to obtain a plant with increased callus initiation and quality when compared to the callus initiation frequency of the second plant.

15. The method of claim 14 wherein the first maize parent is Hi-II.

16. The method of claim 14 wherein the first maize parent is A188.

17. The method of claim 14 wherein the first maize parent is H99.

18. The method of claim 14, further comprising taking DNA from cells obtained from said cross or from cells of later filial generations of said cross and hybridizing with one or more markers located in a group consisting of bin 3.02, 3.03, 3.04, 3.05 and 3.06.

19. A method of breeding a maize plant with increased transformability comprising a) crossing a first maize plant and a second maize plant wherein said first plant has a higher transformation rate than said second plant; b) taking DNA from cells obtained from said cross or from cells of later filial generations of said cross; c) hybridizing said DNA one or more markers, identified in Table 12, and; d) selecting a maize plant with increased transformability when compared to the transformability rate of the second plant.

20. The method of claim 19 wherein the first maize parent is Hi-II.

21. The method of claim 19 wherein the first maize parent is A188.

22. The method of claim 19 wherein the first maize parent is H99.


SEQUENCE LISTING (32 sequences)

SEQ ID NO: 1 - DNA, 22 bp, Zea mays
gctccacatctgctttccctgt

SEQ ID NO: 2 - DNA, 22 bp, Zea mays
tgctccctttgcgcttttagag

SEQ ID NO: 3 - DNA, 21 bp, Zea mays
gtcgacctctccatatcacag

SEQ ID NO: 4 - DNA, 19 bp, Zea mays
gctgctgcatgcataagaa

SEQ ID NO: 5 - DNA, 22 bp, Zea mays
tccttcaaaggttcaaaggaca

SEQ ID NO: 6 - DNA, 21 bp, Zea mays
atgttatgaaaccgtggctga

SEQ ID NO: 7 - DNA, 19 bp, Zea mays
catgaccacgaccatgagc

SEQ ID NO: 8 - DNA, 18 bp, Zea mays
gcaggcgtctccaccttt

SEQ ID NO: 9 - DNA, 22 bp, DNA
gcggtctctcttcctcttcttt

SEQ ID NO: 10 - DNA, 19 bp, Zea mays
acgaggggaaggagacgtt

SEQ ID NO: 11 - DNA, 18 bp, Zea mays
taagcagaggctcgtggc

SEQ ID NO: 12 - DNA, 22 bp, Zea mays
cggctcctacttcatgtacgtc

SEQ ID NO: 13 - DNA, 20 bp, Zea Mays
ggtgctgagagagagggaga

SEQ ID NO: 14 - DNA, 18 bp, Zea mays
ctcgctgttgccttcaaa

SEQ ID NO: 15 - DNA, 18 bp, Zea mays
ggtgaactggggaacgac

SEQ ID NO: 16 - DNA, 21 bp, Zea mays
ctgttgtacaagctccatcgg

SEQ ID NO: 17 - DNA, 22 bp, Zea mays
cattgctttgcttctctttccc

SEQ ID NO: 18 - DNA, 21 bp, Zea mays
tttgattgagctcgattcgtc

SEQ ID NO: 19 - DNA, 18 bp, Zea mays
tcggcatcttacgggctt

SEQ ID NO: 20 - DNA, 18 bp, Zea mays
cgacgcacgcagactttt

SEQ ID NO: 21 - DNA, 19 bp, Zea mays
tgtcgtagtcgcggagaaa

SEQ ID NO: 22 - DNA, 18 bp, Zea mays
taaacgcgcgagtggagt

SEQ ID NO: 23 - DNA, 18 bp, Zea mays
aagttcgggacaccaccg

SEQ ID NO: 24 - DNA, 18 bp, Zea mays
gctgttgcccatgacgat

SEQ ID NO: 25 - DNA, 18 bp, Zea mays
catggtctgccagatcgc

SEQ ID NO: 26 - DNA, 18 bp, Zea mays
gctgctcaggttgttgcc

SEQ ID NO: 27 - DNA, 19 bp, Zea mays
aacgaccagagagacacgg

SEQ ID NO: 28 - DNA, 18 bp, Zea mays
ccgcccgcatagaggata

SEQ ID NO: 29 - DNA, 18 bp, Zea mays
ccggcagatgtttcgatg

SEQ ID NO: 30 - DNA, 18 bp, Zea mays
gaggaaaggatcggacgc

SEQ ID NO: 31 - DNA, 18 bp, Zea mays
gacaagggcgacaagtgg

SEQ ID NO: 32 - DNA, 22 bp, Zea mays
aacataccaaagcagagcaacc